(19) United States
(12) Patent Application Publication
(10) Pub. No.: US 2023/0391855 A1
(43) Pub. Date: Dec. 7, 2023

(54) CHIMERIC ANTI-SARS-COV2 NUCLEOPROTEIN ANTIBODIES

(71) Applicant: LA JOLLA INSTITUTE FOR IMMUNOLOGY, La Jolla, CA (US)

(72) Inventors: Erica Ollmann SAPHIRE, La Jolla, CA (US); Kathryn HASTIE, La Jolla, CA (US); Sara LANDERAS-BUENO, La Jolla, CA (US); Stephanie HARKINS, La Jolla, CA (US)

(21) Appl. No.: 18/249,523

(22) PCT Filed: Oct. 21, 2021
(86) PCT No.: PCT/US2021/056116

§ 371 Date: Apr. 18, 2023

Related U.S. Application Data
(60) Provisional application No. 63/114,918, filed on Nov. 17, 2020.
(60) Provisional application No. 63/105,855, filed on Oct. 26, 2020.
(60) Provisional application No. 63/094,565, filed on Oct. 21, 2020.

(51) Int. Cl. C07K 16/10; G01N 33/68

(52) U.S. Cl. CPC C07K 16/1003; G01N 33/6854; C07K 2317/51; C07K 2317/515

(57) ABSTRACT

Compositions and methods are disclosed for diagnosing, treating, and/or preventing coronavirus infection and related pathologies. The compositions include antibodies having certain defined heavy and light chain sequences that recognize the coronavirus nucleoprotein or mutants thereof. In embodiments, the coronavirus is SAR-CoV-2.

### CROSS-REFERENCE TO RELATED APPLICATIONS

This application claims priority to, and the benefit of, U.S. Provisional Patent Application Ser. No. 63/114,918 filed Nov. 17, 2020, entitled “CHIMERIC ANTI-SARS-COV2 NUCLEOPROTEIN ANTIBODIES”, and claims priority to, and the benefit of, U.S. Provisional Patent Application Ser. No. 63/105,855 filed Oct. 26, 2020, entitled “CHIMERIC ANTI-SARS-COV2 NUCLEOPROTEIN ANTIBODIES”, and claims priority to, and the benefit of, U.S. Provisional Patent Application Ser. No. 63/094,565 filed Oct. 21, 2020, entitled “CHIMERIC ANTI-SARS-COV2 NUCLEOPROTEIN ANTIBODIES”, the entire contents of which are incorporated herein by reference in their entirety.

### TECHNICAL FIELD

The present application relates to antibodies, and particularly to antibodies that recognize SARS-CoV-2 nucleoprotein.

### BACKGROUND

Effective countermeasures against the emergence and expansion of the 2019-Novel Coronavirus (SARS-CoV-2) require the development of new tools for detection, diagnosis, monitoring and treatment.

The worldwide spread of SARS-CoV-2 in the human population resulted in the ongoing COVID-19 pandemic that has already caused more than 241 million infections and more than 4.9 million deaths resulting from SARS-CoV-2 or related diseases and conditions. As such, the aforementioned tools are urgently required.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, an aspect of the present disclosure relates to an antibody composition that recognizes a coronavirus nucleoprotein or mutants thereof. In an aspect, the antibody composition comprises a heavy chain having at least 80%, or at least 85%, or at least 90%, or at least 95% sequence identity with any one of SEQ ID NOs: 1-24. In embodiments, the heavy chain comprises any one of SEQ ID NOs: 1-24.

In another aspect, an antibody composition is disclosed comprising a light chain having at least 80%, or at least 85%, or at least 90%, or at least 95% sequence identity with any one of SEQ ID NOs: 25-48. In embodiments, the light chain comprises any one of SEQ ID NOs: 25-48.

In another aspect, an antibody composition is disclosed comprising a heavy chain having at least 80%, or at least 85%, or at least 90%, or at least 95% sequence identity with any one of SEQ ID NOs: 49-61. In embodiments, the heavy chain comprises any one of SEQ ID NOs: 49-61.

In another aspect, an antibody composition is disclosed comprising a light chain having at least 80%, or at least 85%, or at least 90%, or at least 95% sequence identity with any one of SEQ ID NOs: 62-74. In embodiments, the light chain comprises any one of SEQ ID NOs: 62-74.

In another aspect, an antibody composition is disclosed comprising a heavy chain having at least 80%, or at least 85%, or at least 90%, or at least 95% sequence identity with any one of SEQ ID NOs: 1-24; and a light chain having at least 80%, or at least 85%, or at least 90%, or at least 95% sequence identity with any one of SEQ ID NOs: 25-48. In embodiments, the heavy chain comprises any one of SEQ ID NOs: 1-24; and the light chain comprises any one of SEQ ID NOs: 25-48.

In another aspect, an antibody composition is disclosed comprising a heavy chain having at least 80%, or at least 85%, or at least 90%, or at least 95% sequence identity with any one of SEQ ID NOs: 49-61; and a light chain having at least 80%, or at least 85%, or at least 90%, or at least 95% sequence identity with any one of SEQ ID NOs: 62-74. In embodiments, the heavy chain comprises any one of SEQ ID NOs: 49-61; and the light chain comprises any one of SEQ ID NOs: 62-74.

In another aspect, a method of treating an individual infected with a coronavirus is disclosed. The method involves administering to the individual an effective amount of an antibody composition as detailed herein.

In another aspect, a method of preventing a coronavirus infection in an individual is disclosed. The method involves administering to the individual an effective amount of an antibody composition as detailed herein.

In another aspect, a method of identifying a coronavirus in an individual is disclosed. The method involves isolating a biological sample from the individual; incubating the biological sample with an antibody composition as detailed herein; and detecting a biological interaction between the biological sample and the antibody composition.

In another aspect, a method of detecting a coronavirus in a sample is disclosed. The method involves isolating a biological sample; incubating the biological sample with an antibody composition as detailed herein; and detecting a biological interaction between the biological sample and the antibody composition.

In another aspect, a kid for identifying a coronavirus in a biological sample is disclosed. The kit includes an antibody composition as detailed herein; and instructions of use.

In certain aspects, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages).

In another aspect, an enzyme-linked immunosorbent assay (ELISA) test kit is disclosed. The ELISA test kit includes an antibody composition as detailed herein; and instructions of use.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE FIGURES

In the present application:

FIG. 1 demonstrates certain ELISA binding data as detailed herein.

FIG. 2 demonstrates certain ELISA binding data as detailed herein.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

### Overview of Disclosure

The present disclosure describes antibodies that recognize SARS-CoV-2 nucleoprotein. The disclosure also describes certain methods of treatment, both for therapeutic and prophylactic purposes, as well as diagnostics capable of detecting the presence of SARS-CoV-2 and methods of diagnosing.

### Definitions and Interpretation

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

If and as used herein, the term “administering”, when used in relation to an expression vector, nucleic acid molecule, or a delivery vehicle (such as a chitosan nanoparticle) to a cell, refers to transducing, transfecting, electroporation, translocating, fusing, phagocytosing, shooting or ballistic methods, etc., i.e., any means by which a protein or nucleic acid can be transported across a cell membrane and preferably into the nucleus of a cell.

Unless otherwise indicated, a particular amino acid sequence that is recited herein also implicitly encompasses conservatively modified variants. As a non-limiting example, any of the disclosed amino acid sequences for portions of an anti-SARS-CoV-2 nucleoprotein antibody implicitly encompass conservatively modified variants.

If and as used herein, the terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The polypeptide sequences are displayed herein in the conventional N-terminal to C-terminal orientation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and 0-phosphoserine. The expression “amino acid analogs” refers to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

As used herein, the term “CDR” refers to a complementary determine region of an antibody. A CDR is part of a variable chain of an antibody that binds to an antigen.

As used herein, the phrase “consensus sequence” refers to a sequence that results from a comparison of at least two sequences which contains all possible amino acid residues at each position of the at least two sequences.

If and as used herein, the term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon in an amino acid herein, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid and nucleic acid sequences, individual substitutions, deletions or additions that alter, add or delete a single amino acid or-nucleotide or a small percentage of amino acids or nucleotides in the sequence create a “conservatively modified variant,” where the alteration results in the substitution of an amino acid with a chemically similar amino acid. Conservative substitution tables providing functionally similar amino acids are well known in the art.

For example, the following groups each contain amino acids that are conservative substitutions for one another (see, e.g., Creighton, Proteins (1984) W.H. Freeman, New York, pages 6-20, for a discussion of amino acid properties):

Alanine (A), Glycine (G)
Serine (S), Threonine (T)
Aspartic acid (D), Glutamic acid (E)
Asparagine (N), Glutamine (Q)
Cysteine (C), Methionine (M)
Arginine (E), Lysine (K), Histidine (H)
Isoleucine (I), Leucine (L), Valine (V)
Phenylalanine (F), Tyrosine (Y), Tryptophan (W)

In light of the present disclosure, in particular in view of the experimental data described in the examples of the present text, the person of skill will readily understand which amino acid may be substituted, deleted or added to a given sequence to create a conservatively modified variant comprising an amino acid sequence which is at least at least 95%, or at least 96%, or at least 97%, or at least 98%, or at least 99%, identical to one or more amino acid sequence set forth in the table above without undue effort.

If and used herein, the terms “treating” or “treatment” refers to a process by which an infection, such as infection with a coronavirus, or a disease or the symptoms of an infection or a disease associated with a viral strain are prevented, alleviated or completely eliminated. As used herein, the term “prevented” or “preventing” refers to a process by which an infection or a disease or symptoms of an infection or a disease associated with a virus, such as a coronavirus, are averted prior to infection. In certain aspects, the coronavirus is SARS, MERS, 229E (alpha), NL63 (alpha), (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages).

If and as used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

If and used herein, the expression “an acceptable carrier” refers to a vehicle for containing a compound that can be administered to a subject without significant adverse effects.

As used herein, the term “adjuvant” means a substance added to the composition of the invention to increase the composition's immunogenicity. The mechanism of how an adjuvant operates is not entirely known. Some adjuvants are believed to enhance the immune response (humoral and/or cellular response) by slowly releasing the antigen, while other adjuvants are strongly immunogenic in their own right and are believed to function synergistically.

With respect to the present disclosure, an adjuvant may be selected from aluminum hydroxide or mineral oil, and a stimulator of immune responses, such as Bordatella pertussis or Mycobacterium tuberculosis derived proteins. Suitable adjuvants are commercially available as, for example, Freund's Incomplete Adjuvant and Complete Adjuvant (Pifco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes; biodegradable microspheres; and Quil A. Suitable adjuvants also include, but are not limited to, toll-like receptor (TLR) agonists, particularly toll-like receptor type 4 (TLR-4) agonists (e.g., monophosphoryl lipid A (MPL), synthetic lipid A, lipid A mimetics or analogs), aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, oil-in-water emulsions, MF59, and squalene. In some embodiments, the adjuvants are not bacterially-derived exotoxins. In an embodiment, adjuvants may include adjuvants which stimulate a Thl type response such as 3DMPL or QS21. Adjuvants may also include certain synthetic polymers such as poly amino acids and co-polymers of amino acids, saponin, paraffin oil, and muramyl dipeptide. Adjuvants also encompass genetic adjuvants such as immunomodulatory molecules encoded in a co-inoculated DNA, or as CpG oligonucleotides. The co-inoculated DNA can be in the same plasmid construct as the plasmid immunogen or in a separate DNA vector. The reader can refer to Vaccines (Basel). 2015 June; 3(2): 320-343 for further examples of suitable adjuvants.

If and used herein, the terms “determining,” “measuring,” “evaluating,” “assessing,” and “assaying” generally refer to any form of measurement, and include determining if an element is present or not in a biological sample. These terms include both quantitative and/or qualitative determinations, which both require sample processing and transformation steps of the biological sample. Assessing may be relative or absolute. The phrase “assessing the presence of” can include determining the amount of something present, as well as determining whether it is present or absent.

If and used herein, the expression “biological sample” includes, in the present disclosure, any biological sample that is suspected of comprising a T cell, such as for example but without being limited thereto, blood and fractions thereof, urine, excreta, semen, seminal fluid, seminal plasma, prostatic fluid, pre-ejaculatory fluid (Cowper's fluid), pleural effusion, tears, saliva, sputum, sweat, biopsy, ascites, amniotic fluid, lymph, vaginal secretions, endometrial secretions, gastrointestinal secretions, bronchial secretions, breast secretions, and the like. In one non-limiting embodiment, a herein described biological sample can be obtained by any known technique, for example by drawing, by non-invasive techniques, or from sample collections or banks, etc.

If and used herein, the expression “treatment” includes inducing, enhancing, or sustaining an immune response against a viral infection or symptoms associated thereto. For example, the treatment may induce, increase, promote or stimulate anti-viral virus activity of immune system cells in a subject following the treatment. In non-limiting examples, the immune system cells may include adaptive immune cells, such as T cells, including CD4⁺ T cells, CD8⁺ T cells, and/or B cells, or innate immune cells, such as macrophages and/or neutrophils.

If and used herein, the expression “therapeutically effective amount” may include the amount necessary to allow the component or composition to which it refers to perform its immunological role without causing overly negative effects in the host to which the component or composition is administered. The exact amount of the components to be used or the composition to be administered will vary according to factors such as the type of condition being treated, the type and age of the subject to be treated, the mode of administration, as well as the other ingredients in the composition.

As used herein, the term “virus” generally refers to a coronavirus or any mutant form thereof. As used herein, the term “SARS-CoV-2” refers to all phylogenetic samples of SARS-CoV-2 genomes, as well as any and all mutant strains thereof. As used herein, the term “nucleoprotein” refers to a protein expressed by a coronavirus, and includes, without limitation, reference to SARS-CoV-2 Nucleocapsid Protein (Severe acute respiratory syndrome coronavirus 2 nucleocapsid, 2019 novel coronavirus nucleoprotein, SARS-CoV-2 NP, SARS-CoV-2 N protein, COVID-19).

Other examples of implementations will become apparent to the person skilled in the art in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

Any and all references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

### Detailed Description of Aspects and Embodiments of the Disclosure

As embodied and broadly described herein, an aspect of the present disclosure relates to an antibody composition comprising a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 1-24. In embodiments, the heavy chain comprises any one of SEQ ID NOs: 1-24.

In another aspect, an antibody composition is disclosed comprising a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 25-48. In embodiments, the light chain comprises any one of SEQ ID NOs: 25-48.

In another aspect, an antibody composition is disclosed comprising a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 49-61. In embodiments, the heavy chain comprises any one of SEQ ID NOs: 49-61.

In another aspect, an antibody composition is disclosed comprising a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 62-74. In embodiments, the light chain comprises any one of SEQ ID NOs: 62-74.

In another aspect, an antibody composition is disclosed comprising a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 1-24; and a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 25-48. In embodiments, the heavy chain comprises any one of SEQ ID NOs: 1-24; and the light chain comprises any one of SEQ ID NOs: 25-48.

In another aspect, an antibody composition is disclosed comprising a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 49-61; and a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 62-74. In embodiments, the heavy chain comprises any one of SEQ ID NOs: 49-61; and the light chain comprises any one of SEQ ID NOs: 62-74.

In another aspect, an antibody composition is disclosed comprising a CDR of a heavy chain sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 75-98. In embodiments, the CDR heavy chain comprises any one of SEQ ID NOs: 75-98.

In In another aspect, an antibody composition is disclosed comprising a CDR of a light chain sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 99-122. In embodiments, the CDR light chain comprises any one of SEQ ID NOs: 99-122.

In another aspect, an antibody composition is disclosed comprising a CDR of a heavy chain sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 123-135. In embodiments, the CDR heavy chain comprises any one of SEQ ID NOs: 123-135.

In another aspect, an antibody composition is disclosed comprising a CDR of a light chain sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 136-148. In embodiments, the CDR light chain comprises any one of SEQ ID NOs: 136-148.

In another aspect, an antibody composition is disclosed comprising a CDR of a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 75-98; and a CDR of a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 99-122. In embodiments, the CDR heavy chain comprises any one of SEQ ID NOs: 74-98; and the CDR light chain comprises any one of SEQ ID NOs: 99-122.

In another aspect, an antibody composition is disclosed comprising a CDR of a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 123-135; and a CDR of a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 136-148. In embodiments, the CDR heavy chain comprises any one of SEQ ID NOs: 123-135; and the CDR light chain comprises any one of SEQ ID NOs: 136-148.

In another aspect, an antibody composition is disclosed comprising a consensus sequence of a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 149. In embodiments, the heavy chain comprises SEQ ID NO: 149.

In another aspect, an antibody composition is disclosed comprising a consensus sequence of a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 150. In embodiments, the light chain comprises SEQ ID NO: 150.

In another aspect, an antibody composition is disclosed comprising a consensus sequence of a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 151. In embodiments, the heavy chain comprises SEQ ID NO: 151.

In another aspect, an antibody composition is disclosed comprising a consensus sequence of a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 152. In embodiments, the light chain comprises SEQ ID NO: 152.

In another aspect, an antibody composition is disclosed comprising a consensus sequence of a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 149; and a consensus sequence of light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 150. In embodiments, the consensus heavy chain comprises SEQ ID NO: 149; and the consensus light chain comprises SEQ ID NO: 150.

In another aspect, an antibody composition is disclosed comprising a consensus sequence of a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 151; and a consensus sequence of light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 152. In embodiments, the consensus heavy chain comprises SEQ ID NO: 151; and the consensus light chain comprises SEQ ID NO: 152.

In another aspect, an antibody composition is disclosed comprising a consensus sequence of a heavy chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 153. In embodiments, the consensus heavy chain CDR sequence comprises SEQ ID NO: 153.

In another aspect, an antibody composition is disclosed comprising a consensus sequence of a light chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 154. In embodiments, the consensus light chain CDR sequence comprises SEQ ID NO: 154.

In another aspect, an antibody composition is disclosed comprising a consensus sequence of a heavy chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 155. In embodiments, the consensus heavy chain CDR sequence comprises SEQ ID NO: 155.

In another aspect, an antibody composition is disclosed comprising a consensus sequence of a light chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 156. In embodiments, the consensus light chain CDR sequence comprises SEQ ID NO: 156.

In another aspect, an antibody composition is disclosed comprising a consensus sequence of a heavy chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 153; and a consensus sequence of a light chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 154. In embodiments the consensus heavy chain CDR sequence comprises SEQ ID NO: 153; and the consensus sequence light chain CDR sequence comprises SEQ ID NO: 154.

In another aspect, an antibody composition is disclosed comprising a consensus sequence of a heavy chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 155; and a consensus sequence of a light chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 156. In embodiments the consensus heavy chain CDR sequence comprises SEQ ID NO: 155; and the consensus sequence light chain CDR sequence comprises SEQ ID NO: 156.

The above-mentioned antibody compositions also include any and all variants, modifications, homologues, derivatives or subsequences thereof.

In another aspect, a method of treating an individual infected with a coronavirus is disclosed. The method involves administering to the individual an effective amount of an antibody composition as detailed herein. In certain aspects, the coronavirus is SARS-CoV-2.

In another aspect, a method of preventing coronavirus infection in an individual is disclosed. The method involves administering to the individual an effective amount of an antibody composition as detailed herein.

In another aspect, a method of identifying coronavirus in an individual is disclosed. The method involves isolating a biological sample from the individual; incubating the biological sample with an antibody composition as detailed herein; and detecting a biological interaction between the biological sample and the antibody composition.

In another aspect, a kit for identifying coronavirus in a biological sample is disclosed. The kit includes an antibody composition as detailed herein; and instructions of use.

In another aspect, an enzyme-linked immunosorbent assay (ELISA) test kit is disclosed. The ELISA test kit includes an antibody composition as detailed herein; and instructions of use.

In accordance with the invention, treatment methods are provided that include therapeutic (following infection with a coronavirus) and prophylactic (prior to coronavirus exposure, infection or pathology) methods. For example, therapeutic and prophylactic methods of treating a subject for a viral infection include treatment of a subject having or at risk of having a viral infection or pathology, treating a subject with a viral infection, and methods of protecting a subject from a viral infection, to decrease or reduce the probability of a viral infection in a subject, to decrease or reduce susceptibility of a subject to a viral infection, or to inhibit or prevent a viral infection in a subject, and to decrease, reduce, inhibit or suppress transmission of a virus from a host to a subject.

Such methods include the administration of the antibodies disclosed herein, or nucleic acids encoding the antibodies disclosed herein, to therapeutically or prophylactically treat a subject having or at risk of having a virus infection or pathology. Accordingly, methods can treat the virus infection or pathology, or provide the subject with protection from infection (e.g., prophylactic protection).

In certain aspects, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages). In particular aspects, the coronavirus is SARS-CoV-2 or a variant thereof.

In particular embodiments, one or more disorders, diseases, physiological conditions, pathologies and symptoms associated with or caused by SARS-CoV-2 viral infection or pathology will respond to treatment. In particular methods embodiments, treatment methods reduce, decrease, suppress, limit, control or inhibit viral numbers or titer; reduce, decrease, suppress, limit, control or inhibit pathogen proliferation or replication; reduce, decrease, suppress, limit, control or inhibit the amount of a pathogen protein; or reduce, decrease, suppress, limit, control or inhibit the amount of a viral nucleic acid. In additional particular methods embodiments, treatment methods include an amount of an antibody composition, subsequence or portion thereof sufficient to increase, induce, enhance, augment, promote or stimulate an immune response against a virus; increase, induce, enhance, augment, promote or stimulate viral clearance or removal; or decrease, reduce, inhibit, suppress, prevent, control, or limit transmission of a particular virus to a subject (e.g., transmission from a host to a subject). In further particular methods embodiments, treatment methods include an amount of an antibody composition, subsequence or portion thereof sufficient to protect a subject from a viral infection or pathology, or reduce, decrease, limit, control or inhibit susceptibility to viral infection or pathology.

Methods of the present disclosure include treatment methods, which result in any therapeutic or beneficial effect. In various methods embodiments, viral infection, proliferation or pathogenesis is reduced, decreased, inhibited, limited, delayed or prevented, or a method decreases, reduces, inhibits, suppresses, prevents, controls or limits one or more adverse (e.g., physical) symptoms, disorders, illnesses, diseases or complications caused by or associated with a viral infection, proliferation or replication, or pathology. In additional various particular embodiments, treatment methods include reducing, decreasing, inhibiting, delaying or preventing onset, progression, frequency, duration, severity, probability or susceptibility of one or more adverse symptoms, disorders, illnesses, diseases or complications caused by or associated with a particular viral infection, proliferation or replication, or pathology. In further various particular embodiments, treatment methods include improving, accelerating, facilitating, enhancing, augmenting, or hastening recovery of a subject from a particular viral infection or pathogenesis, or one or more adverse symptoms, disorders, illnesses, diseases or complications caused by or associated with a viral infection, proliferation or replication, or pathology. In yet additional various embodiments, treatment methods include stabilizing infection, proliferation, replication, pathogenesis, or an adverse symptom, disorder, illness, disease or complication caused by or associated with a viral infection, proliferation or replication, or pathology, or decreasing, reducing, inhibiting, suppressing, limiting or controlling transmission of a virus from and to an uninfected subject.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease or complication caused by or associated with a viral infection, proliferation or replication, or pathology. Thus, a satisfactory clinical endpoint is achieved when there is an incremental improvement or a partial reduction in an adverse symptom, disorder, illness, disease or complication caused by or associated with a viral infection, proliferation or replication, or pathology, or an inhibition, decrease, reduction, suppression, prevention, limit or control of worsening or progression of one or more adverse symptoms, disorders, illnesses, diseases or complications caused by or associated with a particular viral infection, viral numbers, titers, proliferation or replication, viral protein or nucleic acid, or viral pathology, over a short or long duration (hours, days, weeks, months, etc.).

A therapeutic or beneficial effect also includes reducing or eliminating the need, dosage frequency or amount of a second active such as another drug or other agent (e.g., anti-viral) used for treating a subject having or at risk of having a viral infection or pathology. For example, reducing an amount of an adjunct therapy, for example, a reduction or decrease of a treatment for a viral infection or pathology, or a vaccination or immunization protocol is considered a beneficial effect. In addition, reducing or decreasing an amount of a viral antigen used for vaccination or immunization of a subject to provide protection to the subject is considered a beneficial effect.

Adverse symptoms and complications associated with a viral infection and pathology include, but are not limited to for example, e.g., fever, rash, headache, cough, shortness of breath or difficulty breathing, chills (including shaking associated with chills), sore throat, loss of taste or smell, tiredness, difficulty breathing, pain behind the eyes, conjunctivitis, muscle or joint pain, nausea, vomiting, loss of appetite, or secondary infection. Other symptoms of a viral infection or pathogenesis are known to one of skill in the art and treatment thereof in accordance with the invention is provided. Thus, the aforementioned symptoms and complications are treatable in accordance with the present disclosure.

Adverse symptoms and complications associated with viral infections and pathologies can include, but are not limited to, for example, e.g., fever, rash, headache, cough, tiredness, difficulty breathing, pain including pain behind the eyes, stomach pain, muscle or joint pain, weakness, fatigue, conjunctivitis, nausea, vomiting, diarrhea, loss of appetite, hemorrhaging, bleeding, bruising, or secondary infection. Other symptoms of viral infections or pathogenesis are known to one of skill in the art and treatment thereof in accordance with the invention is provided. Thus, the aforementioned symptoms and complications are treatable in accordance with the present disclosure.

Methods, uses and compositions of the present disclosure include administration of the antibody compositions to a subject prior to contact, exposure or infection by a particular virus, administration prior to, substantially contemporaneously with or after a subject has been contacted by, exposed to or infected with a particular virus, and administration prior to, substantially contemporaneously with or after a particular virus pathology or development of one or more adverse symptoms, disorders, illness or diseases caused by or associated with a particular viral infection, or pathology. A subject infected with a particular virus may have an infection over a period of 1-5, 5-10, 10-20, 20-30, 30-50, 50-100 hours, days, months, or years.

Compositions and uses and methods of the present disclosure can be combined with any compound, agent, drug, treatment or other therapeutic regimen or protocol having a desired therapeutic, beneficial, additive, synergistic or complementary activity or effect.

Combination methods and use embodiments include, for example, second actives such as anti-pathogen drugs, such as protease inhibitors, reverse transcriptase inhibitors, virus fusion inhibitors and virus entry inhibitors, antibodies to pathogen proteins, live or attenuated pathogen, or a nucleic acid encoding all or a portion (e.g., an epitope) of any protein or proteinaceous pathogen antigen, immune stimulating agents, etc., and include contact with, administration in vitro or in vivo, with another compound, agent, treatment or therapeutic regimen appropriate for pathogen infection, vaccination or immunization.

Methods of the present disclosure also include, among other things, methods that result in a reduced need or use of another compound, agent, drug, therapeutic regimen, treatment protocol, process, or remedy. For example, for a particular viral infection or pathology, vaccination or immunization, a method of the present disclosure has a therapeutic benefit if in a given subject a less frequent or reduced dose or elimination of an anti-viral treatment results. Thus, in accordance with the invention, methods of reducing need or use of a treatment or therapy for a particular viral infection or pathology, or vaccination or immunization, are provided.

Methods of the present disclosure include methods of diagnosing a viral infection in a subject. In embodiments, the viral infection comprises an infection with coronavirus. In an aspect, the method of diagnosing a viral infection comprises obtaining a biological sample from a subject and contacting the biological sample with any of the antibody compositions described herein. In embodiments, the biological sample comprises a tissue sample from a subject. In embodiments, the biological sample comprises a blood sample from a subject. In embodiments, the biological sample derives from a nasal swab of a subject. In embodiments, the biological sample derives from a throat swab of a subject.

Methods of the present methods include methods of detecting a viral protein. In embodiments, the method comprises detecting a coronavirus protein. In embodiments, the method comprises detecting a coronavirus nucleoprotein.

In certain aspects, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages).

In embodiments, the methods of diagnosing a viral infection and the methods of detecting a viral protein comprise contacting the sample with any of the antibody compositions described herein. In embodiments, the antibody compositions described herein comprise at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one or more of SEQ ID NOs: 1-156. In embodiments, the antibody compositions described herein comprise any one or more of SEQ ID NOs: 1-156.

In embodiments, the antibody compositions described herein comprise a detectable moiety. Examples of the detectable moieties include but are not limited to a radiolabel, a fluorescent label, an enzymatic label, biotin, chromophore, or an ECL label.

In embodiments, the methods of diagnosing a viral infection and methods of detecting a viral protein comprise using antibody-based assays. In embodiments, the antibody-based assay is an ELISA assay. In embodiments, the antibody-based assay is a radioimmunoassay. In embodiments, the antibody-based assay is fluoroimmunoassay. In embodiments, the antibody-based assay is a chemiluminescenceimmunoassay.

In an aspect, the present disclosure also provides diagnostic kits comprising any one or more of any of the antibody compositions described herein. In embodiments, the antibody compositions described herein comprise at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one or more of SEQ ID NOs: 1-156. In embodiments, the antibody compositions described herein comprise any one or more of SEQ ID NOs: 1-156.

In embodiments, the antibody compositions in the kit bind to a protein of a virus. In embodiments, the antibody compositions in the kit bind to a protein of a coronavirus. In embodiments, the antibody compositions in the kit bind to a nucleoprotein of a coronavirus. In embodiments, the antibody compositions in the kit contain a detectable moiety. In embodiments, the detectable moiety comprises a radiolabel, a fluorescent label, an enzymatic label, biotin, chromophore, or an ECL label. In certain aspects, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineage).

In embodiments, the diagnostic kit contains primary antibodies and secondary antibodies. In embodiments, the primary antibodies comprise any of the antibody compositions described herein. In embodiments, the secondary antibodies are capable of binding the primary antibodies and contain a suitable detectable label.

Without limiting any of the foregoing, the following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local.

The compositions of the present disclosure may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

For instance, the composition of the present disclosure may be administered in the form of an injectable preparation, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection, by infusion or per os. Suitable dosages will vary, depending upon factors such as the amount of each of the components in the composition, the desired effect (short or long term), the route of administration, the age and the weight of the subject to be treated. Any other methods well known in the art may be used for administering the composition of the present disclosure.

The antibody compositions of the present disclosure may be formulated as a dry powder (i.e., in lyophilized form). Freeze-drying (also referred to as lyophilisation) is often used for preservation and storage of biologically active material because of the low temperature exposure during drying. Lyophilizing the composition can result in a more stable composition.

In certain embodiments, the composition of the present disclosure may be formulated as a liquid (e.g., aqueous formulation), e.g., as syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Where the composition of the present disclosure is intended for delivery to the respiratory (e.g., nasal) mucosa, typically it is formulated as an aqueous solution for administration as an aerosol or nasal drops, or alternatively, as a dry powder, e.g., for rapid deposition within the nasal passage. Compositions for administration as nasal drops may contain one or more excipients of the type usually included in such compositions, for example preservatives, viscosity adjusting agents, tonicity adjusting agents, buffering agents, and the like. Viscosity agents can be microcrystalline cellulose, chitosan, starches, polysaccharides, and the like. Compositions for administration as dry powder may also contain one or more excipients usually included in such compositions, for example, mucoadhesive agents, bulking agents, and agents to deliver appropriate powder flow and size characteristics. Bulking and powder flow and size agents may include mannitol, sucrose, trehalose, and xylitol.

### EXAMPLES

### Example 1. ELISA Binding Data

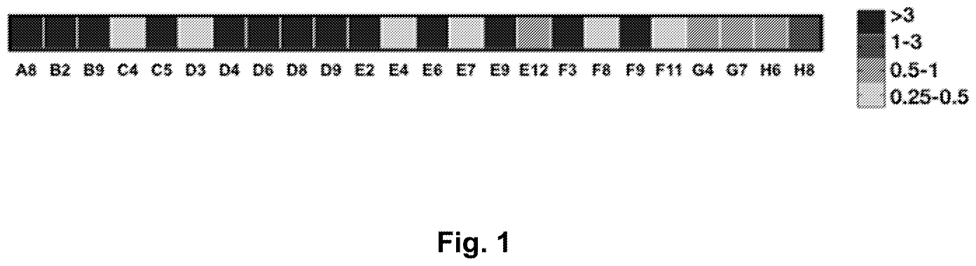

SARS-CoV-2 N protein was coated on ELISA microtiters plates. Supernatants from small-scale (2 mL) antibody test expressions was captured overnight and binding analyzed with an anti-mouse IgG-HRP secondary. 14 total antibodies bound to N with an OD>1. These antibodies included A8, B2, B9, C5, D4, D6, D8, D9, E2, E6, E9, F3, F9, H8. Another 10 antibodies bound to N with an OD between 0.25 and 1. These antibodies included C4, D3, E4, E7, E12, F8, F11, G4, G7, and H6. The data is summarized in FIG. 1.

Heavy chain sequences corresponding to the tested sequences are described below:

Heavy Chains
NP1_HC_A8
(SEQ ID NO: 1)
DVQLQESGPGLVKPSQSLSLTCTVTGYSITSDYAWNWIRQFPGNKLEWMGYI
SYSGSTSYNPSLKSRISITRDTSKNQFFLQLNSVTTEDTATYYCARGRDGYLYYFDYWGQ
GTTLTV
NP1_HC_B2
(SEQ ID NO: 2)
EVQLQQSGAELVKPGASVKLSCTASGINIKDIYIHWVKQRPEQGLEWIGRIDP
ANGNIKYDPKFQDKATMTADTSSNIAYLQLSSLTSEDTAVYYCARWLQRFLDYWGQGT
SVTV
NP1_HC_B9
(SEQ ID NO: 3)
QVQLQQSGAELMKPGASVKISCKATGYTFSDYWIVWVKQRPGHGLEWIGEI
LPGSGNTNYNEKFKGKATFTADASSNTAYMELSSLSSEDSAVYYCASRLLLRRYFDVWG
AGTPVTV
NP1_HC_C4
(SEQ ID NO: 4)
QVQLQQSGPELVRPGVSVKISCKGSGYTFTDYVMHWVKQSRAKSLEWIGVI
STYSGNTNYNQKFKGKATMTVDKSSSTAYMELARLTSEDSAIYYCARGTGDFDYWGQG
TTLTV
NP1_HC_C5
(SEQ ID NO: 5)
EVQLVESGGGLVKPGGSLKLSCAASGFIFSDYYMFWVRQTPEKRLEWVATIS
DGGRYTYYPDSVQGRFTISRHNAKNNLYLQMSSLKSEDTAIYYCARDRYDGWGSDFDV
WGAGTTVTV
NP1_HC_D3
(SEQ ID NO: 6)
QVQLQQSGPEVVRPGVSEKISCKGSGYTFTDYALHWVKQSHAKSLEWIGVIS
TYSGTTNYNQNFKGKATMAVDKSSRTAYMELARLTSDDSAIYYCASGGNYGLDYWGQ
GTSVTV
NP1_HC_D4
(SEQ ID NO: 7)
EVQLVESGGDLVKPGGSLKLSCAASGFTFSDYYMSWVRQTPEKRLEWVATI
SDGGRYTYYPDSVKGRFTISRDNAKNNLYLQMGSLKSEDTAMYYCARGSTYGNPLDY
WGQGTTLTVS
NP1_HC_D6
(SEQ ID NO: 8)
QVTLKESGPGILQPSQTLSLTCSFSGFSLSTSGTGVSWIRQPSGKGLEWLAHIY
WDDDKRYNPSLKSRLTLSKDTSTNQVFLKITSVDTADTATYYCARSNYGYDFDYWGPG
TTLTV
NP1_HC_D8
(SEQ ID NO: 9)
QVQLQQSGPELVKPGASVKMSCKASGYTFTDYVINWVKQRTGQGLEWIGEI
YPESDNTFYNEKFRGRATLTADKSSNTAYMQLSSLTSGDSAVYFCARRGFYDGNYVRYF
DVWGAGTTVTV
NP1_HC_D9
(SEQ ID NO: 10)
QVQLQQSGPELVKPGASVKISCKASGYSFTGYFMNWVKQSHGRSLEWIGRI
NSYNGDTFYNQKFKGKATLTVDKSSSTAHMELLSLTSEDSAVYYCGLTTVVATKYFDY
WGQGTTLTV
NP1_HC_E2
(SEQ ID NO: 11)
QVQLQQPGAELVKPGAPVKLSCKASGYTFTSYWMNWVKQRPGRDLEWIGR
IDPSDSETHYNQKFKDKATLTVDKSSSTAYIQLSSLTSEDSAVYYCSREVATATKGVFSW
FAYWGQGTLVTV
NP1_HC_E4
(SEQ ID NO: 12)
QVQLKESGPGLVAPSQSLSITCTVSGFSLTSYGVHWVRQPPGKGLEWLVVIW
SDGSTTYNSALKSRLSISKDNSKSQVFLKMNSLQTDDTAMYYCARNYYGSSYYAMDYW
GQGTSVTVS
NP1_HC_E6
(SEQ ID NO: 13)
QVQLQQSGPELVKPGASVKISCKASGYSFTAYFMNWVKQSHGKSLEWIGRI
NPYNGDTFYNQKFKGKATLTVDKSSSTAHMDLLSLTSEDSAVYYCGLTTLVATKYFDY
WGQGTTLTVS
NP1_HC_E7
(SEQ ID NO: 14)
EVQLQQSGAELVKPGASVKLSCTASGINIKDIYIHWVKQRPEQGLEWIGRIDP
ANGNIKYDPKFQDKATMTADTSSNIAYLQLSSLTSEDTAVYYCARWLQRFLDYWGQGT
SVTVS
NP1_HC_E9
(SEQ ID NO: 15)
QVQLQQSGPELVKPGTLVKISCKASGYTFTSYDINWVKQRPGQGLEWIGWIY
PGDGSTKYNEKFKGKATLTADTSSSTAYMQLNSLTSENSAVYFCARGLVGAMDYWGQ
GTSVTVS
NP1_HC_E12
(SEQ ID NO: 16)
QIQLVQSGPELKKPGETVKISCKASGYTFTDYSIHWVKQAPGKGLKWMGWI
NTYTGEPTYADDFKGRFAFSLETSATTAYLQINNLKNEDSATYFCARSHDNYDNHFDYW
GQGTTLTV
NP1_HC_F3
(SEQ ID NO: 17)
EVQLVESGGGLVQPGGSLKLSCAASGFTFSSYTMSWVRQTPEKRLEWVAFIS
YSGTNVYYPDTVKGRFTISRDVAKNALYLQMTSLKSEDTALYFCARHGRFTTVVEGYFD
VWGAGTTVTVS
NP1_HC_F8
(SEQ ID NO: 18)
QVQLQQSGPELVRPGVSVKISCKGSGYTFTDYAMHWVKQSHAKSLEWIGVI
STYYGNTNYNQKFKGKATMTVDKSSSTAYMELARLTSEDSAIYYCARGITTAPFDYWG
QGTTLTVS
NP1_HC_F9
(SEQ ID NO: 19)
EVQLVESGGGLVRPGGSLKLSCAASGFTFSDYYIYWVRQTPEKRLEWVATIS
DGGRYTYYPDSVKGRFTISRDNAKNNLYLQMTSLKSEDTAMYYCTRGDDYGGRLAYW
GQGTLVTV
NP1_HC_F11
(SEQ ID NO: 20)
QVQLQQSGAELVRPGASVKISCKTFGYSFTYHHINWVKQRPGQGLDWIGYIN
PYNDYSGYDHNFKGKATLTVDKSSSTAYMELSSLTSEDSAIYYCTAERGSYAMDYWGQ
GTS
NP1_HC_G4
(SEQ ID NO: 21)
DVQLQESGPGLVKPSQSLSLTCTVTGYSITSDSAWNWIRQFPRNKLEWMGYI
SYSGATTYNPSLKSRFSITRDTSKNQFFLQLNSVTTEDTATYYCAREGIHYAMDYWGQG
TSVTVS
NP1_HC_G7
(SEQ ID NO: 22)
EVQLQQSGAELVRPGALVKLSCKASGFDIKDFYMNWVRQRPEQGLEWIGWI
DPENGNIKYNPKFQGKANITAGTSSNTAYLQLSSLTSEDTAVYYCAREGLSSMITTVYYY
AMDYWGQGTSVTVS
NP1_HC_H6
(SEQ ID NO: 23)
DVQLQESGPGLVKPSQSLSLTCTVTGYSSTSDYAWNWIRQFPGNKLEWMGY
VSYSGSTSYNPSLKSRNSITRDTSKNQFFLQLNSVTTEDTATYYCARGRDGYLYYFDYW
GQGTTLTVS
NP1_HC_H8
(SEQ ID NO: 24)
QVQLQQSGAELVKPGASVKISCKATGYTLSSDWIEWVKQRPGHGLEWIGEIL
PGSDNTDYNEKFTGKATFTADTSSNTAYMQLSSLTSEDSAVYYCARRKFLRRYFDVWG
AGTTVTVS

Light chain sequences corresponding to the tested sequences are described below:

Light Chains
NP1_kLC_A8
(SEQ ID NO: 25)
DIQMTQTTSSLSASLEDRVTISCSASQGIRNYLNWYQQKPDGTVKLLI
FFTSTLHSGVPSRFSGSGSGTDYSLTISNLEPEDIATYYCHQYSKLPY
TFGGGTKL
NP1_kLC_B2
(SEQ ID NO: 26)
DIVMTQAAPSIPVTPGESVSISCRSNQSLLHSSGNTFLYWFLQRPGQS
PQLLIYRMSNLASGVPDRFSGSGSGTAFTLRISRVEAEDVGVYYCMQH
LEYPLTFGGGTKL
NP1_kLC_B9
(SEQ ID NO: 27)
DIVLTQSPASLAVSLGQRATISCKASQSVDYDGDSYMNWYQQKPGQPP
KLLIYAASNLESGIPARFSGSGSGTDFTLNIHPVEEEDAATYYCQQSN
EDPFTFGGGTKL
NP1_kLC_C4
(SEQ ID NO: 28)
DIVMTQSPSSLAVSVGEKVTMSCKSGQSLLYSSNQKNYLAWYQQKPGQ
SPKLLIYWASTRESGVPDRFTGSGSGTDFTLTISSVKAEDLAVYFCQQ
YYSYPWTFGGGTKLEIKRT
NP1_kLC_C5
(SEQ ID NO: 29)
ENVLTQSPAIMSASLGERVTMTCTASSSVNSNYLHWYQQKPGSSPKLW
IYSTSNLASGVPTRFSGSGSGTSYSLTISSMEAEDAATYYCHQYHRSP
RTFGGGTKL
NP1_kLC_D3
(SEQ ID NO: 30)
DIVMTQSPSSLSMSAGEKVTMSCKSSQSLLNSGNQKNYLAWYQQKPGQ
PPKLLIYGASTRESGVPDRFTGSGSGTDFTLTISSVQAEDLAVYYCQN
DHSYPYTFGWGTKLELKRT
NP1_kLC_D4
(SEQ ID NO: 31)
DIQMTQTTSSLSASRGDRVTISCSASQDISNFLNWYQQKPDGTVKLLI
YYTSSLHSGVPSRFSGSGSGTDYSLTISNLEPEDIATYYCQQYSKLPF
TFGSGTKLELKRT
NP1_kLC_D6
(SEQ ID NO: 32)
DIVMTQSQKFMSTSVGDRVSVTCKASQNVGTNVAWYQQKPGQSPKALI
YSASYRYSGVPDRFTGSGSGTDFTLTISNMQSEDLAEYFCQQYNSYPT
FGGGTKLELKRT
NP1_kLC_D8
(SEQ ID NO: 33)
DIQMTQTTSSMYASLGERVTITCKASQDIKSYLSWYQQKPWKSPKTLI
YYATSLADGVPSRFSGSGSGQDYSLTISSLESDDTATYYCLQHGESPL
TFGAGTKLELKRT
NP1_kLC_D9
(SEQ ID NO: 34)
DIVMTQSPSSLTVSVGEKVTMSCKSSQSLLYSSNQKNFLAWYQQKPGQ
SPKLLIYWASTRESGVPDRFTGSGSGTDFTLTISSVKAEDLAVYYCQQ
YYSYPLTFGAGTKLELKRT
NP1_kLC_E2
(SEQ ID NO: 35)
DIQMTQTTSSLSASLGDRVTISCSASQDIRDYLNWYQQKPDGTVKLLI
YYTSILHSGVPSRFSGSGSGTDYSLTISNLASEDIATYYCQQYSKLPW
TFGGGTKLEIKRT
NP1_kLC_E4
(SEQ ID NO: 36)
DIKMTQSPSSMYASLGERVTITCKASQDIKSYLSWYQQKPWKSPKTLI
YYATSLADGVPSRFSGSGSGQDYSLTISSLESDDTATYYCLQHGESPF
TFGSGTKLELKRT
NP1_kLC_E6
(SEQ ID NO: 37)
DIVMTQSPSSLAVSVGEKVTMSCKSSQSLLYSSNQKNFLAWYQQKPGQ
SPKLLIYWASTRESGVPDRFTGSGSGTDFTLTISSVKAEDLAVYYCQQ
YFSYPLTFGAGTKLELKRT
NP1_kLC_E7
(SEQ ID NO: 38)
DVVMTQTPLTLSVTIGQPASISCKSSQSLLDSDGKTYLNWLLQRPGQS
PKRLIYLVSKLDSGVPDRFTGSGSGTDFTLKISRVEAEDLGVYYCWQG
THLWTFGGGTKLEIKRT
NP1_kLC_E9
(SEQ ID NO: 39)
DIVMTQSQKFMSTSVGDRVSVTCKASQNVLNNVAWYQQKPGQSPKALI
YSASYRYSGVPDRFTGSGSGTDFTLTISNVQSEDLAEYFCQQYNSYPL
TFGDGTKLELKRT
NP1_kLC_E12
(SEQ ID NO: 40)
ENVLTQSPALMSASPGGKVTMTCSASSSVSYMYWYQQKPRSSPKPWIY
LTSNLASGVPARFSGSGSGTSYSLTISSMEAEDAATYYCQQWSINPYT
FGGGTKLEIKRT
NP1_kLC_F3
(SEQ ID NO: 41)
DIVMTQSPATLSVTPGDRVSLSCRASQSISDYLHWYQQKSHESPRLLI
KYVSQSISGIPSRFSGSGSGSDFTLSINSVEPEDVGIYYCQNGHSFPW
TFGGGTKLEIKRT
NP1_kLC_F8
(SEQ ID NO: 42)
DIVMTQSHKFMSTSVGDRVSITCKASQDVSTAVAWYQQKPGQSPKLLI
YWESTRHTGVPDRFTGSGSGTDLTLTISNVQSEDLADYFCQQYSSYPY
TFGGGTKLEIKRT
NP1_kLC_F9
(SEQ ID NO: 43)
DIQMTQTTSSQSASLGDRVTINCSASQGISNYLNWYQQKPDGTVKLLI
YYTSSLHSGVPSRFSGSGSGTEYSLTISNLEPEDIATYYCQQYSKLPY
TFGGGTKLEIKRT
NP1_kLC_F11
(SEQ ID NO: 44)
DVVMTQTPLSLPVSLGDQASISCRSSQSLVHSNGNTYLHWYLQKPGQS
PKLLIYKVSNRFSGVPDRFSGSGSGTDFTLKISRVEAEDLGVYFCSQS
THVPFTFGSGTKLEIKRT
NP1_kLC_G4
(SEQ ID NO: 45)
DIKMTQSPSSMYASLGERVTITCKASQDIKSYLSWYQQKPWKSPKTLI
YYATSLADGVPSRFSGSGSGQDYSLTISSLESDDTATYYCLQHGESPL
TFGAGTKLEIKRT
NP1_kLC_G7
(SEQ ID NO: 46)
DIVMTQAAASIPVSPGESVSISCRSNQSLLHSSGITFLYWFLQRPGQS
PHLLIYRMSNLASGVPNRFSGSGSGTAFTLRISKVEAEDVGVYYCMQH
LEYPLTFGGGTKLEIKRT
NP1_kLC_H6
(SEQ ID NO: 47)
DIQMTQTTSSLSASLGDRVTISCSASQDINNNLNWYQQKPDVTVKLLI
YYTSSLHSGVPSRLSGSGSGTDYSLTISNLEPEDIATYYCQQYSKFPY
TFGGGTKLEIKRT
NP1_KLC H8
(SEQ ID NO: 48)
DIQMTQTTSSLSASLGDRVTISCSASQDINNNLNWYQQKPDGTVKLLI
YYTSSLHSGVPSRLSGSGSGTDYSLTISNLEPEDIATYYCQQYSKFPY
TFGGGTKLEIKRT

### Example 2. ELISA Binding Data

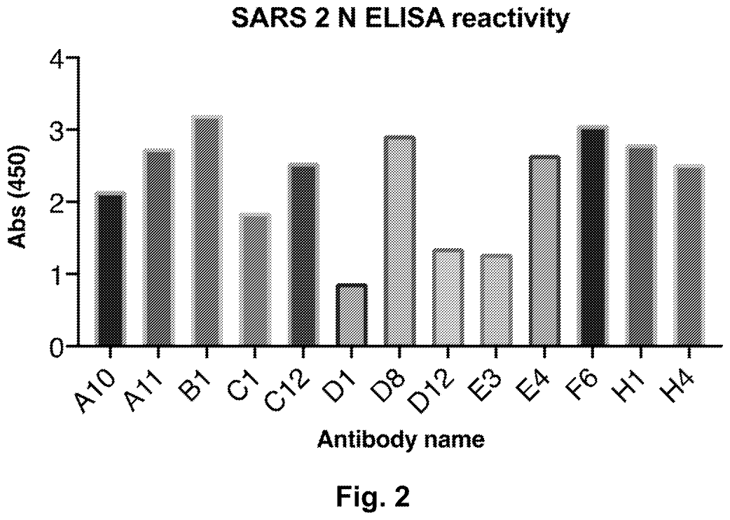

SARS-CoV-2 N protein was coated on ELISA microtiters plates. Supernatants from small-scale (2 mL) antibody test expressions was captured overnight and binding analyzed with an 15 anti-mouse IgG-HRP secondary. Nine antibodies had an absorbance over 2. These antibodies included A10, A11, B1, C12, D8, E4, F6, H1, and H4. Four antibodies had an absorbance between 0.5 and 2. These antibodies included Cl, D1, D12, and E3. The data is summarized in FIG. 2.

Heavy chain sequences corresponding to the tested sequences are described below:

Heavy Chains
NP2_HC_A10
(SEQ ID NO: 49)
EVQLQESGAELVRPGSSVKISCKVSGYTFSNYWMNWVKQRPGQGLEW
IGQIYPGDDSTYYNGKFKDKAKMTADKSSSTAYIQLSSLTSEDSAVY
FCARRPLFYAMDYWGQGTSVTVS
NP2_HC_A11
(SEQ ID NO: 50)
EVQLQESGPELKKPGETVKISCKASGYTFTNYGMNWVKQAPGKGLKW
MGWINTYTGEPTYADDFKGRFAFSLDTSTTTANLQINNLTNDDMATY
FCARTRETWGNVDSAYWGQGTLVTV
NP2_HC_B1
(SEQ ID NO: 51)
EVQLQESGPERKKPGETVKISCKASGYSLTYHSMHWVKQAPGKGLKW
MGWINTETGEPTYADDFKGRFAFSLETSASTAYLQINNLKNEDTATY
FCTRKHGNSYYYAMDYWGQGTSVTVS
NP2_HC-C1
(SEQ ID NO: 52)
EVQLQESGPELKKPGETVKISCKASGYTFTNYGMNWVKQAPGKGLKW
MGWINTYTGEPTYADDFKGRFAFSLDTSTTTANLQINNLTNDDMATY
FCARTRETWGNVDSAYWGQGTLVTV
NP2_HC_C12
(SEQ ID NO: 53)
EVQLQESGGGLVRPGGSLKLSCAASGFTFSDYYMYWVRQTPEKRLEW
VATISDGGRYSYYPDSVKGRFTVSRDNAKNNLYLQMSSLKSEDTAMY
YCVRDRYDGWGSDFDVWGAGTTVTVS
NP2_HC_D1
(SEQ ID NO: 54)
EVQLQESGPELVKPGASVKMSCKASGYTFTRYVMHWVKQKPGQGLEW
IGYINPYNDGTKYNEKFKGKATLTSDKSSSTAYMELRSLTSEDSAVY
YCARLGPRYYFDYWGQGTTLTVS
NP2_HC_D8
(SEQ ID NO: 55)
EVQLQESGAELMKPGASVKISCKATGSTFSSYWIEWVKQRPGHGLEW
IGEILPGSGNTNYNEKFKAKATFTADTASNTAYMQLTSLTSEDSAVH
YCARRWFLRRYFDVWGAGTTVTVS
NP2_HC_D12
(SEQ ID NO: 56)
EVQLQESGGGLVKPGGSLKLSCAASGFTFSDYYMYWVRQTPEKRLEW
VATISDGGRYTYYPDSVQGRFTISRDNAKNNLYLQMSSLKSEDTAMY
YCARDRYDGWGSDFDVWGAGTTVTVS
NP2_HC_E3
(SEQ ID NO: 57)
EVQLQESGGGLVKPGGSLKLSCAASGFTFSDYYMYWVRQTPEKRLEW
VATISDGGRYTYYPDSVQGRFTISRDNAKNNLYLQMSSLKSEDTAMY
YCARDRYDGLGSDFDVWGAGTTVTVS
NP2_HC_E4
(SEQ ID NO: 58)
EVQLQESGGDSVKPGGSLKLSCAASGFTFSTYTMSWVRQTPEKRLEW
VATISSGGTYTYYPDSVKGRFTISRDNAKSTLYLQMSSLKSEDTALY
HCTRDRDYYVSSRGFAYWGQGTLVTVS
NP2_HC_F6
(SEQ ID NO: 59)
EVQLQESGTELMKPGASVKISCKATGSTFSSYWIEWVKQRPEHGLEW
IGEILPGSGNTNYNEKFRAKATFTADTASNTAYMQLSSLTSEDSAVH
YCARRWFLRRYFDVWGAGTTVTVS
NP2_HC_H1
(SEQ ID NO: 60)
EVQLQESGGGLVQPGRSLKLSCAASGFTFSDYYLFWFRQTPEKRLEW
VATISDGGRYTYYLDSVKGRFTISRDNAKNKVYLQMSSLKSEDTAMY
YCARDNANYGGYFDYWGQGTTLTVS
NP2_HC-H4
(SEQ ID NO: 61)
EVQLQESGGGLVKPGGSLKLSCAASGFIFSDYYMFWVRQTPEKRLEW
VATISDGGRYTYYPDSVQGRFTISRDNAKNNLYLQTSSLKSEDTAIY
YCARDRYDGWGSDFDVWGAGTTVTVS

Light chain sequences corresponding to the tested sequences are described below:

Light Chains
NP2_kLC_A10
(SEQ ID NO: 62)
DIQMTQSPSSLAMSVGQKVTMSCKSSQSLLNSSNQKNYLAWYQQKPG
QSPKLLVYFASTRESGVPDRFIGSGSGTDFTLTISSVQAEDLADYFC
QQHYSAPPTFGAGTKLEIKR
NP2_kLC_A11
(SEQ ID NO: 63)
DIQMTQSPSSLSVSVGETVTITCRASENVYSNLAWYQQKQGKSPQLR
VYAATDLADGVPSRFSGRGSGTQYSLKINNLQSEDFGTYYCQHVWGT
PWTFGGGTKLEIKR
NP2_kLC_B1
(SEQ ID NO: 64)
DIQMTQSPSSLSVSLGDRVTISCSVSQDISDYLNWYQQKPDGTVKLL
IYFTSSLHSGVPSRFSGSGSGTDYSLTISNLEPEDLATYYCQQYSKL
PFTFGSGTKLEIKR
NP2_kLC_C1
(SEQ ID NO: 65)
DVVMTQTPLTLSVTIGQPASISCKSSQTLLDSDGKTYLNWLLQRPGQ
SPKRLIYLVSELDSGVPDRFTGSGSGTDFTLKISRVEAEDLGIYYCW
QGTHFPRTFGGGTKLEIKR
NP2_kLC_C12
(SEQ ID NO: 66)
DVQITQSPSSMYASLGERVTITCKASQDIKSYLSWYQQKPWKSPKTL
IYYATSLADGVPSRFSGSGSGQDYSLTISSLESDDTATYYCLQHGES
PWTFGGGTKLEIKR
NP2_kLC_D1
(SEQ ID NO: 67)
DVQITQSPSSLSVSVGETVTITCRASENIYSNLAWYQQKQGKSPQLL
VYAATNLADGVPSRFSGSGSGTHYSLKINNLQSEDFGSYYCQHFWGT
PWTFGGGTKLEIKR
NP2_kLC_D8
(SEQ ID NO: 68)
ENVLTQSPASLAVSLGQKATISCKASQSVDYDGESYMNWYQQKPGQP
PKLLIYAASNLESGIPARFSGSGSGTDFTLNIHPAEEEDVATYYCQQ
SNGDPFTFGGGTKLEIKR
NP2_kLC_D12
(SEQ ID NO: 69)
ENVLTQSPASLAVSLGQRATISCRASESVGSYGNSFMHWYQQKPGQP
PKLLIYRAPNLESGIPARFSGSGSRTDFTLTINPVEADDVATYYCQQ
SNEDPYTFGGGTKLELKR
NP2_kLC_E3
(SEQ ID NO: 70)
DVQITQSPSSMYASLGERVTITCKASQDIKSYLSWYQQKPWKSPKTL
IYYATSLADGVPSRLSGSGSGQDYSLTISSLESDDTATYYCLQHGES
PLTFGAGTKLEIKR
NP2_kLC_E4
(SEQ ID NO: 71)
DVVMTQTPLTLSVTIGQPASISCKSSQSLLDSDGKTHLNWLLQRPGQ
SPKRLIYLVSKLDSGVPDRFTGSGSGTDFTLKISRVEAEDLGVYYCW
QETHFPWTFGGGTKLELKR
NP2_kLC_F6
(SEQ ID NO: 72)
DVQITQSPASLAVSLGQRATISCKASQSVDYDGDSYMNWYQQKPGQP
PKLLIYAASNLESGIPARFSGSGSGTDFTLNIHPVEEEDAATYYCQQ
SNEDPFTFGGGTKLEIKR
NP2_kLC_H1
(SEQ ID NO: 73)
DIQMTQSPSSMYASLGERVTITCKASQDIKSYLSWYQQKPWKSPKTL
IYYATSLADGVPSRFSGSGSGQDYSLTISSLESDDTATYYCLQHGES
PFTFGSGTKLEIKR
NP2_kLC_H4
(SEQ ID NO: 74)
ENVLTQSPAIMSASPGEKVTITCSASSSVSYMHWFQQKPGTSPKLWI
YSTSNLASGVPARFSGSGSGTSYSLTISRMEAEDAATYYCQQRSSYP
LTFGAGTKLEL

### Example 3. CDR Sequences of Heavy Chain and Light Chain Sequences

The antibody sequences described in Examples 1 and 2 were further analyzed to identify the CDR sequences of the heavy chain and light chain sequences.

Heavy Chain CDR sequences of SEQ ID NOs: 1-24

Each of the heavy chain sequences of SEQ ID NOs: 1-24 were analyzed to identify CDR sequences. SEQ ID NOs: 75-98 constitute CDR sequences within SEQ ID NOs: 1-24.

NP1_HC_A8
(SEQ ID NO: 75)
CARGRDGYLYYFDYW
(CDR sequence within SEQ ID NO: 1)
NP1_HC_B2
(SEQ ID NO: 76)
CARWLQRFLDYW
(CDR sequence within SEQ ID NO: 2)
NP1_HC_B9
(SEQ ID NO: 77)
CASRLLLRRYFDVW
(CDR sequence within SEQ ID NO: 3)
NP1_HC_C4
(SEQ ID NO: 78)
CARGTGDFDYW
(CDR sequence within SEQ ID NO: 4)
NP1_HC_C5
(SEQ ID NO: 79)
CARDRYDGWGSDFDVW
(CDR sequence within SEQ ID NO: 5)
NP1_HC_D3
(SEQ ID NO: 80)
CASGGNYGLDYW
(CDR sequence within SEQ ID NO: 6)
NP1_HC_D4
(SEQ ID NO: 81)
CARGSTYGNPLDYW
(CDR sequence within SEQ ID NO: 7)
NP1_HC_D6
(SEQ ID NO: 82)
CARSNYGYDFDYW
(CDR sequence within SEQ ID NO: 8)
NP1_HC_D8
(SEQ ID NO: 83)
CARRGFYDGNYVRYFDVW
(CDR sequence within SEQ ID NO: 9)
NP1_HC_D9
(SEQ ID NO: 84)
CGLTTVVATKYFDYW
(CDR sequence within SEQ ID NO: 10)
NP1_HC_E2
(SEQ ID NO: 85)
CSREVATATKGVFSWFAYW
(CDR sequence within SEQ ID NO: 11)
NP1_HC_E4
(SEQ ID NO: 86)
CARNYYGSSYYAMDYW
(CDR sequence within SEQ ID NO: 12)
NP1_HC_E6
(SEQ ID NO: 87)
CGLTTLVATKYFDYW
(CDR sequence within SEQ ID NO: 13)
NP1_HC_E7
(SEQ ID NO: 88)
CARWLQRFLDYW
(CDR sequence within SEQ ID NO: 14)
NP1_HC_E9
(SEQ ID NO: 89)
CARGLVGAMDYW
(CDR sequence within SEQ ID NO: 15)
NP1_HC_E12
(SEQ ID NO: 90)
CARSHDNYDNHFDYW
(CDR sequence within SEQ ID NO: 16)
NP1_HC_F3
(SEQ ID NO: 91)
CARHGRFTTVVEGYFDVW
(CDR sequence within SEQ ID NO: 17)
NP1_HC_F8
(SEQ ID NO: 92)
CARGITTAPFDYW
(CDR sequence within SEQ ID NO: 18)
NP1_HC_F9
(SEQ ID NO: 93)
CTRGDDYGGRLAYW
(CDR sequence within SEQ ID NO: 19)
NP1_HC_F11
(SEQ ID NO: 94)
CTAERGSYAMDYW
(CDR sequence within SEQ ID NO: 20)
NP1_HC_G4
(SEQ ID NO: 95)
CAREGIHYAMDYW
(CDR sequence within SEQ ID NO: 21)
NP1_HC_G7
(SEQ ID NO: 96)
CAREGLSSMITTVYYYAMDYW
(CDR sequence within SEQ ID NO: 22)
NP1_HC_H6
(SEQ ID NO: 97)
CARGRDGYLYYFDYW
(CDR sequence within SEQ ID NO: 23)
NP1_HC_H8
(SEQ ID NO: 98)
CARRKFLRRYFDVW
(CDR sequence within SEQ ID NO: 24)

Light Chain CDR sequences of SEQ ID NOs: 25-48

Each of the light chain sequences of SEQ ID NOs: 25-48 were analyzed to identify CDR sequences. SEQ ID NOs: 99-122 constitute CDR sequences within SEQ ID NOs: 25-48.

NP1_kLC_A8
(SEQ ID NO: 99)
CHQYSKLPYTF
(CDR sequence within SEQ ID NO: 25)
NP1_kLC_B2
(SEQ ID NO: 100)
CMQHLEYPLTF
(CDR sequence within SEQ ID NO: 26)
NP1_kLC_B9
(SEQ ID NO: 101)
CQQSNEDPFTF
(CDR sequence within SEQ ID NO: 27)
NP1_kLC_C4
(SEQ ID NO: 102)
CQQYYSYPWTF
(CDR sequence within SEQ ID NO: 28)
NP1_kLC_C5
(SEQ ID NO: 103)
CHQYHRSPRTF
(CDR sequence within SEQ ID NO: 29)
NP1_kLC_D3
(SEQ ID NO: 104)
CQNDHSYPYTF
(CDR sequence within SEQ ID NO: 30)
NP1_KLC_D4
(SEQ ID NO: 105)
QQYSKLPFTF
(CDR sequence within SEQ ID NO: 31)
NP1_kLC_D6
(SEQ ID NO: 106)
CQQYNSYPTF
(CDR sequence within SEQ ID NO: 32)
NP1_kLC_D8
(SEQ ID NO: 107)
CLQHGESPLTF
(CDR sequence within SEQ ID NO: 33)
NP1_kLC_D9
(SEQ ID NO: 108)
CQQYYSYPLTF
(CDR sequence within SEQ ID NO: 34)
NP1_kLC_E2
(SEQ ID NO: 109)
CQQYSKLPWTF
(CDR sequence within SEQ ID NO: 35)
NP1_kLC_E4
(SEQ ID NO: 110)
CLQHGESPFTF
(CDR sequence within SEQ ID NO: 36)
NP1_kLC_E6
(SEQ ID NO: 111)
CQQYFSYPLTF
(CDR sequence within SEQ ID NO: 37)
NP1_kLC_E7
(SEQ ID NO: 112)
CWQGTHLWTF
(CDR sequence within SEQ ID NO: 38)
NP1_kLC_E9
(SEQ ID NO: 113)
CQQYNSYPLTF
(CDR sequence within SEQ ID NO: 39)
NP1_kLC_E12
(SEQ ID NO: 114)
CQQWSINPYTF
(CDR sequence within SEQ ID NO: 40)
NP1_kLC_F3
(SEQ ID NO: 115)
CQNGHSFPWTF
(CDR sequence within SEQ ID NO: 41)
NP1_kLC_F8
(SEQ ID NO: 116)
CQQYSSYPYTF
(CDR sequence within SEQ ID NO: 42)
NP1_kLC_F9
(SEQ ID NO: 117)
CQQYSKLPYTF
(CDR sequence within SEQ ID NO: 43)
NP1_kLC_F11
(SEQ ID NO: 118)
CSQSTHVPFTF
(CDR sequence within SEQ ID NO: 44)
NP1_kLC_G4
(SEQ ID NO: 119)
CLQHGESPLTF
(CDR sequence within SEQ ID NO: 45)
NP1_kLC_G7
(SEQ ID NO: 120)
CMQHLEYPLTF
(CDR sequence within SEQ ID NO: 46)
NP1_kLC_H6
(SEQ ID NO: 121)
CQQYSKFPYTF
(CDR sequence within SEQ ID NO: 47)
NP1_kLC_H8
(SEQ ID NO: 122)
CQQYSKFPYTF
(CDR sequence within SEQ ID NO: 48)

Heavy Chain CDR sequences of SEQ ID NOs: 49-61

Each of the heavy chain sequences of SEQ ID NOs: 49-61 were analyzed to identify CDR sequences. SEQ ID NOs: 123-135 constitute CDR sequences within SEQ ID NOs: 49-61.

NP2_HC_A10
(SEQ ID NO: 123)
CARRPLFYAMDYW
(CDR sequence within SEQ ID NO: 49)
NP2_HC_A11
(SEQ ID NO: 124)
CARTRETWGNVDSAYW
(CDR sequence within SEQ ID NO: 50)
NP2_HC_B1
(SEQ ID NO: 125)
CTRKHGNSYYYAMDYW
(CDR sequence within SEQ ID NO: 51)
NP2_HC-C1
(SEQ ID NO: 126)
CARTRETWGNVDSAYW
(CDR sequence within SEQ ID NO: 52)
NP2_HC_C12
(SEQ ID NO: 127)
CVRDRYDGWGSDFDVW
(CDR sequence within SEQ ID NO: 53)
NP2_HC_D1
(SEQ ID NO: 128)
CARLGPRYYFDYW
(CDR sequence within SEQ ID NO: 54)
NP2_HC_D8
(SEQ ID NO: 129)
CARRWFLRRYFDVW
(CDR sequence within SEQ ID NO: 55)
NP2_HC_D12
(SEQ ID NO: 130)
CARDRYDGWGSDFDVW
(CDR sequence within SEQ ID NO: 56)
NP2_HC_E3
(SEQ ID NO: 131)
CARDRYDGLGVLTSMSG
(CDR sequence within SEQ ID NO: 57)
NP2_HC_E4
(SEQ ID NO: 132)
CTRDRDYYVSSRGFAYW
(CDR sequence within SEQ ID NO: 58)
NP2_HC_F6
(SEQ ID NO: 133)
CARRWFLRRYFDVW
(CDR sequence within SEQ ID NO: 59)
NP2_HC_H1
(SEQ ID NO: 134)
CARDNANYGGYFDYW
(CDR sequence within SEQ ID NO: 60)
NP2_HC-H4
(SEQ ID NO: 135)
CARDRYDGWGSDFDVW
(CDR sequence within SEQ ID NO: 61)

Light Chain CDR sequences of SEQ ID NOs: 62-74

Each of the heavy chain sequences of SEQ ID NOs: 62-74 were analyzed to identify CDR sequences. SEQ ID NOs: 136-148 constitute CDR sequences within SEQ ID NOs: 62-74.

NP2_kLC_A10
(SEQ ID NO: 136)
CQQHYSAPPTF
(CDR sequence within SEQ ID NO: 62)
NP2_kLC_A11
(SEQ ID NO: 137)
CQHVWGTPWTF
(CDR sequence within SEQ ID NO: 63)
NP2_kLC_B1
(SEQ ID NO: 138)
CQQYSKLPFTF
(CDR sequence within SEQ ID NO: 64)
NP2_kLC_C1
(SEQ ID NO: 139)
CWQGTHFPRTF
(CDR sequence within SEQ ID NO: 65)
NP2_kLC_C12
(SEQ ID NO: 140)
CLQHGESPWTF
(CDR sequence within SEQ ID NO: 66)
NP2_kLC_D1
(SEQ ID NO: 141)
CQHFWGTPWTF
(CDR sequence within SEQ ID NO: 67)
NP2_kLC_D8
(SEQ ID NO: 142)
CQQSNGDPFTF
(CDR sequence within SEQ ID NO: 68)
NP2_kLC_D12
(SEQ ID NO: 143)
CQQSNEDPYTF
(CDR sequence within SEQ ID NO: 69)
NP2_kLC_E3
(SEQ ID NO: 144)
CLQHGESPLTF
(CDR sequence within SEQ ID NO: 70)
NP2_kLC_E4
(SEQ ID NO: 145)
CWQETHFPWTF
(CDR sequence within SEQ ID NO: 71)
NP2_kLC_F6
(SEQ ID NO: 146)
CQQSNEDPFTF
(CDR sequence within SEQ ID NO: 72)
NP2_kLC_H1
(SEQ ID NO: 147)
CLQHGESPFTF
(CDR sequence within SEQ ID NO: 73)
NP2_kLC_H4
(SEQ ID NO: 148)
CQQRSSYPLTF
(CDR sequence within SEQ ID NO: 74)

### Example 4. Consensus Sequences of the Heavy Chain and Light Chain Sequences

The antibodies were analyzed to identify consensus sequences of the heavy chain and light chain sequences.

Consensus Sequence of the Heavy Chain Sequences of SEQ ID NOs: 1-24

The heavy chains of SEQ ID NOs: 1-24 were analyzed for variability across each amino acid position. SEQ ID NO: 149 represents a consensus sequence based on this analysis.

(SEQ ID NO: 149)
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃
X₂₄X₂₅X₂₆X₂₇X₂₈X₂₉X₃₀X₃₁X₃₂X₃₃X₃₄X₃₅X₃₆X₃₇X₃₈X₃₉X₄₀X₄₁X₄₂X₄₃
X₄₄X₄₅X₄₆X₄₇X₄₈X₄₉X₅₀X₅₁X₅₂X₅₃X₅₄X₅₅X₅₆X₅₇X₅₈X₅₉X₆₀X₆₁X₆₂X₆₃
X₆₄X₆₅X₆₆X₆₇X₆₈X₆₉X₇₀X₇₁X₇₂X₇₃X₇₄X₇₅X₇₆X₇₇X₇₈X₇₉X₈₀X₈₁X₈₂X₈₃
X₈₄X₈₅X₈₆X₈₇X₈₈X₈₉X₉₀X₉₁X₉₂X₉₃X₉₄X₉₅X₉₆X₉₇X₉₈X₉₉X₁₀₀X₁₀₁X₁₀₂
X₁₀₃X₁₀₄X₁₀₅X₁₀₆X₁₀₇X₁₀₈X₁₀₉X₁₁₀X₁₁₁X₁₁₂X₁₁₃X₁₁₄X₁₁₅X₁₁₆X₁₁₇X₁₁₈
X₁₁₉X₁₂₀X₁₂₁X₁₂₂X₁₂₃X₁₂₄×₁₂₅X₁₂₆X₁₂₇X₁₂₈X₁₂₉

wherein,
X₁=D, Q, or E;
X₂=V or I;
X₃=Q or T;
X₄=L;
X₅=Q, K, or V;
X₆=E or Q;
X₇=S or P;
X₈=G;
X₉=P, G, or A;
X₁₀=G, D, or E;
X₁₁=L, I, or V;
X₁₂=V, L, M, or K;
X₁₃=K, A, Q, or R;
X₁₄=P;
X₁₅=S or G;
X₁₆=Q, G, A, T, V, or E;
X₁₇=S, T, L, or P;
X₁₈=L, V, or E;
X₁₉=S or K;
X₂₀=L, I, or M;
X₂₁=T or S;
X₂₂=C;
X₂₃=T, S, A, or K;
X₂₄=V, F, A, G, or T;
X₂₅=T, S, or F;
X₂₆=G;
X₂₇=Y, F, or I;
X₂₈=S, I, T, N, or D;
X₂₉=I, S, L, or F;
X₃₀=S or is absent;
X₃₁=T or is absent;
X₃₂=S, K, or T;
X₃₃=D, G, S, A, or Y, or is absent;
X₃₄=Y, S, T, I, F, D, or H;
X₃₅=A, G, Y, T, W, V, D, F, H, or S;
X₃₆=W, V, M, I, or L;
X₃₇=N, H, S, F, Y, V, or E;
X₃₈=W;
X₃₉=I or V;
X₄₀=R or K;
X₄₁=Q;
X₄₂=F, P, T, R, S, or A;
X₄₃=P, S, T, R, or H;
X₄₄=G, R, E, or A;
X₄₅=N, K, Q, H, or R;
X₄₆=K, G, R, S, or D;
X₄₇=L;
X₄₈=E, D, or K;
X₄₉=W;
X₅₀=M, L, V, or I;
X₅₁=G, V, or A;
X₅₂=Y, V, H, T, F, R, W, or E;
X₅₃=I or V;
X₅₄=S, W, Y, D, L, or N;
X₅₅=P, T, or S, or is absent;
X₅₆=Y, S, W, D, A, E, or G;
X₅₇=S, D, G, N, Y, or T;
X₅₈=G, D, or S;
X₅₉=S, A, D, R, T, N, E, or Y;
X₆₀=T, K, Y, N, I, S, or P;
X₆₁=S, T, R, V, K, N, D, F, H, or G;
X₆₂=Y;
X₆₃=N, Y, D, or A;
X₆₄=P, S, E, Q, H, or D;
X₆₅=D or is absent;
X₆₆=S, A, T, K, N, or D;
X₆₇=L, V, or F;
X₆₈=K, Q, T, or R;
X₆₉=S, G, or D;
X₇₀=R or K;
X₇₁=I, N, F, L, or A;
X₇₂=S, T, N, or A;
X₇₃=I, L, M, or F;
X₇₄=T, S, or A;
X₇₅=R, K, A, V, or L;
X₇₆=D, H, G, or E;
X₇₇=T, N, V, A, or K;
X₇₈=S or A;
X₇₉=K, T, S, or A;
X₈₀=N, S, R, or T;
X₈₁=Q, N, A, I, or T;
X₈₂=F, V, L, or A;
X₈₃=F, Y, or H;
X₈₄=L, M, or I;
X₈₅=Q, K, E, or D;
X₈₆=L, M, or I;
X₈₇=N, T, S, G, A, or L;
X₈₈=S, R, or N;
X₈₉=V or L;
X₉₀=T, Q, D, K, or S;
X₉₁=T, S, or N;
X₉₂=E, D, A, or G;
X₉₃=D or N;
X₉₄=T or S;
X₉₅=A;
X₉₆=T, M, I, L, or V;
X₉₇=Y;
X₉₈=Y or F;
X₉₉=C;
X₁₀₀=T, or A, or is absent;
X₁₀₁=A, T, G, S, or R;
X₁₀₂=R, S, L, or E, or is absent;
X₁₀₃=H, E, R, G, or T, or is absent;
X₁₀₄=G, N, S, D, W, L, K, I, T, or V, or is absent;
X₁₀₅=R, Y, N, L, F, T, V, A, or S, or is absent;
X₁₀₆=D, E, Y, F, Q, S, L, V, or T, or is absent;
X₁₀₇=G, D, S, T, R, or A;
X₁₀₈=Y, I, S, G, T, D, F, M, P, or N, or is absent;
X₁₀₉=L, H, S, V, I, N, or K, or is absent;
X₁₁₀=Y, W, V, T, or G, or is absent;
X₁₁₁=G, E, T, or V, or is absent;
X₁₁₂=Y, A, S, N, G, V, or F, or is absent;
X₁₁₃=A, D, P, R, Y, or S, or is absent;
X₁₁₄=Y, A, or W, or is absent;
X₁₁₅=Y or is absent;
X₁₁₆=A or is absent;
X₁₁₇=F, M, or L;
X₁₁₈=D or A;
X₁₁₉=Y or V;
X₁₂₀=W;
X₁₂₁=G;
X₁₂₂=Q, P, or A;
X₁₂₃=G;
X₁₂₄=T;
X₁₂₅=T, S, L, or P;
X₁₂₆=L, or V, or is absent;
X₁₂₇=T or is absent;
X₁₂₈=V or is absent;
X₁₂₉=S or is absent.

Consensus Sequence of the Light Chain Sequences of SEQ ID NOs: 25-48

The light chains of SEQ ID NOs: 25-48 were analyzed for variability across each amino acid position. SEQ ID NO: 150 represents a consensus sequence based on this analysis.

(SEQ ID NO: 150)
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃
X₂₄X₂₅X₂₆X₂₇X₂₈X₂₉X₃₀X₃₁X₃₂X₃₃X₃₄X₃₅X₃₆X₃₇X₃₈X₃₉X₄₀X₄₁X₄₂X₄₃
X₄₄X₄₅X₄₆X₄₇X₄₈X₄₉X₅₀X₅₁X₅₂X₅₃X₅₄X₅₅X₅₆X₅₇X₅₈X₅₉X₆₀X₆₁X₆₂X₆₃
X₆₄X₆₅X₆₆X₆₇X₆₈X₆₉X₇₀X₇₁X₇₂X₇₃X₇₄X₇₅X₇₆X₇₇X₇₈X₇₉X₈₀X₈₁X₈₂X₈₃
X₈₄X₈₅X₈₆X₈₇X₈₈X₈₉X₉₀X₉₁X₉₂X₉₃X₉₄X₉₅X₉₆X₉₇X₉₈X₉₉X₁₀₀X₁₀₁X₁₀₂
X₁₀₃X₁₀₄X₁₀₅X₁₀₆X₁₀₇X₁₀₈X₁₀₉X₁₁₀X₁₁₁X₁₁₂X₁₁₃X₁₁₄X₁₁₅

wherein,
X₁=D or E;
X₂=I, N, or V;
X₃=Q, K, or V;
X₄=M or L;
X₅=T;
X₆=Q;
X₇=T, S, or A;
X₈=T, P, A, Q, or H;
X₉=S, A, P, L, or K;
X₁₀=S, I, L, T, or F;
X₁₁=L, Q, M, or I;
X₁₂=S, Y, P, A, or T;
X₁₃=A, V, M, or T;
X₁₄=S or T;
X₁₅=L, R, P, I, V, or A;
X₁₆=E or G;
X₁₇=D, E, G, or Q;
X₁₈=R, K, S, P, or Q;
X₁₉=V or A;
X₂₀=T or S;
X₂₁=I, M, V, or L;
X₂₂=S, N, or T;
X₂₃=C;
X₂₄=S, K, T, or R;
X₂₅=A or S;
X₂₆=S, N, or G;
X₂₇=Q or S;
X₂₈=G, D, S, or N;
X₂₉=I, V, or L;
X₃₀=R, S, N, K, L, V, G, or D, or is absent;
X₃₁=S, H, D, Y, N, or T, or is absent;
X₃₂=N, D, S, or A, or is absent;
X₃₃=S, D, N, or G, or is absent;
X₃₄=G, N, or D, or is absent;
X₃₅=Q or is absent;
X₃₆=N, I, or K, or is absent;
X₃₇=T, N, or S, or is absent;
X₃₈=Y, F, or N, or is absent;
X₃₉=L, M, or V;
X₄₀=N, S, H, Y, or A;
X₄₁=W;
X₄₂=Y, F, or L;
X₄₃=Q or L;
X₄₄=Q;
X₄₅=K or R;
X₄₆=P or S;
X₄₇=D, W, G, R, or H;
X₄₈=G, V, K, S, Q, or E;
X₄₉=T, S, or P;
X₅₀=V or P;
X₅₁=K, Q, H, or R;
X₅₂=L, T, P, R, or A;
X₅₃=L or W;
X₅₄=I;
X₅₅=F, Y, or K;
X₅₆=F, Y, S, L, R, K, W, G, or A;
X₅₇=T, A, M, V, or E;
X₅₈=S or T;
X₅₉=T, S, I, N, K, Y, or Q;
X₆₀=L, R, or S;
X₆₁=H, A, D, F, E, Y, or I;
X₆₂=S, D, or T;
X₆₃=G;
X₆₄=V or I;
X₆₅=P;
X₆₆=S, T, A, D, or N;
X₆₇=R;
X₆₈=F or L;
X₆₉=S or T;
X₇₀=G;
X₇₁=S;
X₇₂=G;
X₇₃=S;
X₇₄=G;
X₇₅=T, Q, or S;
X₇₆=D, E, S, or A;
X₇₇=Y, F, or L;
X₇₈=S or T;
X₇₉=L;
X₈₀=T, R, K, S, or N;
X₈₁=I;
X₈₂=S, N, or H;
X₈₃=N, S, R, K, or P;
X₈₄=L, M, or V;
X₈₅=E, A, K, or Q;
X₈₆=P, S, A, or E;
X₈₇=E or D;
X₈₈=D;
X₈₉=I, T, A, V, or L;
X₉₀=A or G;
X₉₁=T, V, E, D, or I;
X₉₂=Y;
X₉₃=Y or F;
X₉₄=C;
X₉₅=H, Q, L, M, W, or S;
X₉₆=Q or N;
X₉₇=Y, H, W, G, S, or D;
X₉₈=S, G, H, L, T, Y, F, or N;
X₉₉=K, E, R, I, H, or S;
X₁₀₀=L, F, S, N, Y, V, or D;
X₁₀₁=P or is absent;
X₁₀₂=Y, F, W, L, or R, or is absent;
X₁₀₃=T;
X₁₀₄=F;
X₁₀₅=G;
X₁₀₆=G, S, A, W, or D;
X₁₀₇=G;
X₁₀₈=T;
X₁₀₉=K;
X₁₁₀=L;
X₁₁₁=E or is absent;
X₁₁₂=L or I, or is absent;
X₁₁₃=K or is absent;
X₁₁₄=R or is absent;
X₁₁₅=T or is absent.

Consensus Sequence of the Heavy Chain Sequences of SEQ ID NOs: 49-61

The heavy chains of SEQ ID NOs: 49-61 were analyzed for variability across each amino acid position. SEQ ID NO: 151 represents a consensus sequence based on this analysis.

(SEQ ID NO: 151)
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃
X₂₄X₂₅X₂₆X₂₇X₂₈X₂₉X₃₀X₃₁X₃₂X₃₃X₃₄X₃₅X₃₆X₃₇X₃₈X₃₉X₄₀X₄₁X₄₂X₄₃
X₄₄X₄₅X₄₆X₄₇X₄₈X₄₉X₅₀X₅₁X₅₂X₅₃X₅₄X₅₅X₅₆X₅₇X₅₈X₅₉X₆₀X₆₁X₆₂X₆₃
X₆₄X₆₅X₆₆X₆₇X₆₈X₆₉X₇₀X₇₁X₇₂X₇₃X₇₄X₇₅X₇₆X₇₇X₇₈X₇₉X₈₀X₈₁X₈₂X₈₃
X₈₄X₈₅X₈₆X₈₇X₈₈X₈₉X₉₀X₉₁X₉₂X₉₃X₉₄X₉₅X₉₆X₉₇X₉₈X₉₉X₁₀₀X₁₀₁X₁₀₂
X₁₀₃X₁₀₄X₁₀₅X₁₀₆X₁₀₇X₁₀₈X₁₀₉X₁₁₀X₁₁₁X₁₁₂X₁₁₃X₁₁₄X₁₁₅X₁₁₆X₁₁₇X₁₁₈
X₁₁₉X₁₂₀X₁₂₁

wherein,
X₁=E;
X₂=V;
X₃=Q;
X₄=L;
X₅=Q;
X₆=E;
X₇═S;
X₈=G;
X₉=A, P, T, or G;
X₁₀=E, G, or D;
X₁₁=L, R, or S;
X₁₂=V, M, or K;
X₁₃=R, K, or Q;
X₁₄=P;
X₁₅=G;
X₁₆=S, A, E, G, or R;
X₁₇=S or T;
X₁₈=V or L;
X₁₉=K;
X₂₀=I, M, or L;
X₂₁=S;
X₂₂=C;
X₂₃=K or A;
X₂₄=V or A;
X₂₅=S or T;
X₂₆=G;
X₂₇=Y, S, or F;
X₂₈=T, S, or I;
X₂₉=F or L;
X₃₀=S or T;
X₃₁=N, R, S, Y, D, or T;
X₃₂=Y or H;
X₃₃=W, V, G, S, Y, or T;
X₃₄=M, I, or L;
X₃₅=N, H, E, Y, F, or S;
X₃₆=W;
X₃₇=V or F;
X₃₈=K or R;
X₃₉=Q;
X₄₀=R, K, A, or T;
X₄₁=P;
X₄₂=G or E;
X₄₃=Q, H, or K;
X₄₄=G or R;
X₄₅=L;
X₄₆=E or K;
X₄₇=W;
X₄₈=I, M, or V;
X₄₉=G or A;
X₅₀=Q, Y, E, W, or T;
X₅₁=I;
X₅₂=Y, N, L, or S;
X₅₃=P, T, D, or S;
X₅₄=G, Y, or E;
X₅₅=D, N, S, T, or G;
X₅₆=D, G, R, or T;
X₅₇=S, G, N, E, or Y;
X₅₈=T, P, or S;
X₅₉=Y, K, N, or T;
X₆₀=Y;
X₆₁=N, A, P, or L;
X₆₂=G, E, or D;
X₆₃=K, D, or S;
X₆₄=F or V;
X₆₅=K, R, or Q;
X₆₆=D, G, or A;
X₆₇=K or R;
X₆₈=A or F;
X₆₉=K, T, or A;
X₇₀=M, L, F, V, or I;
X₇₁=T or S;
X₇₂=A, S, L, or R;
X₇₃=D or E;
X₇₄=K, T, or N;
X₇₅=S or A;
X₇₆=S, T, A, or K;
X₇₇=S, N, or T;
X₇₈=T, N, or K;
X₇₉=A, L, or V;
X₈₀=Y or N;
X₈₁=I, M, or L;
X₈₂=Q or E;
X₈₃=L, I, M, or T;
X₈₄=S, R, T, or N;
X₈₅=S or N;
X₈₆=L;
X₈₇=T or K;
X₈₈=S or N;
X₈₉=E or D;
X₉₀=D;
X₉₁=S, M, or T;
X₉₂=A;
X₉₃=V, T, M, I, or L;
X₉₄=Y or H;
X₉₅=F, Y, or H;
X₉₆=C;
X₉₇=A, T, or V;
X₉₈=R;
X₉₉=L, R, T, K, or D, or is absent;
X₁₀₀=R, G, W, H, or N;
X₁₀₁=D or is absent;
X₁₀₂=P, F, E, G, Y, or A;
X₁₀₃=L, R, T, N, D, or Y;
X₁₀₄=F, Y, R, W, S, G, or V, or is absent;
X₁₀₅=Y, R, G, W, L, or S;
X₁₀₆=A, Y, N, G, or S, or is absent;
X₁₀₇=M, F, V, Y, S, G, or R;
X₁₀₈=D, A, Y, or G;
X₁₀₉=S, M, or F, or is absent;
X₁₁₀=A, or D, or is absent;
X₁₁₁=Y or V;
X₁₁₂=W;
X₁₁₃=G;
X₁₁₄=Q or A;
X₁₁₅=G;
X₁₁₆=T;
X₁₁₇=S, T, or L;
X₁₁₈=V or L;
X₁₁₉=T;
X₁₂₀=V;
X₁₂₁=S or is absent.
Consensus Sequence of the Light Chain Sequences of SEQ ID NOs: 62-74
The light chains of SEQ ID NOs: 62-74 were analyzed for variability across each amino acid position. SEQ ID NO: 152 represents a consensus sequence based on this analysis.

(SEQ ID NO: 152)
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃
X₂₄X₂₅X₂₆X₂₇X₂₈X₂₉X₃₀X₃₁X₃₂X₃₃X₃₄X₃₅X₃₆X₃₇X₃₈X₃₉X₄₀X₄₁X₄₂X₄₃
X₄₄X₄₅X₄₆X₄₇X₄₈X₄₉X₅₀X₅₁X₅₂X₅₃X₅₄X₅₅X₅₆X₅₇X₅₈X₅₉X₆₀X₆₁X₆₂X₆₃
X₆₄X₆₅X₆₆X₆₇X₆₈X₆₉X₇₀X₇₁X₇₂X₇₃X₇₄X₇₅X₇₆X₇₇X₇₈X₇₉X₈₀X₈₁X₈₂X₈₃
X₈₄X₈₅X₈₆X₈₇X₈₈X₈₉X₉₀X₉₁X₉₂X₉₃X₉₄X₉₅X₉₆X₉₇X₉₈X₉₉X₁₀₀X₁₀₁X₁₀₂
X₁₀₃X₁₀₄X₁₀₅X₁₀₆X₁₀₇X₁₀₈X₁₀₉X₁₁₀X₁₁₁X₁₁₂X₁₁₃X₁₁₄

wherein,
X₁=D or E;
X₂=I, V, or N;
X₃=Q or V;
X₄=M, L, or I;
X₅=T;
X₆=Q;
X₇=S or T;
X₈=P;
X₉=S, L, or A;
X₁₀=S, T, or I;
X₁₁=L or M;
X₁₂=A, S, or Y;
X₁₃=M, V, or A;
X₁₄=S or T;
X₁₅=V, I, L, or P;
X₁₆=G;
X₁₇=Q, E, or D;
X₁₈=K, P, R, or T;
X₁₉=V or A;
X₂₀=T or S;
X₂₁=M or I;
X₂₂=S or T;
X₂₃=C;
X₂₄=K, R, or S;
X₂₅=S, A, or V;
X₂₆═S;
X₂₇=Q, E, or S;
X₂₈=S, T, N, or D;
X₂₉=L, V, or I, or is absent;
X₃₀=L, or V, or is absent;
X₃₁=N, D, or G, or is absent;
X₃₂=S, or Y, or is absent;
X₃₃=S, D, or Y, or is absent;
X₃₄=N, or G, or is absent;
X₃₅=Q or is absent;
X₃₆=K, E, D, N, Y, or S, or is absent;
X₃₇=N, T, S, or D;
X₃₈=Y, H, F, or N;
X₃₉=L or M;
X₄₀=A, N, H, or S;
X₄₁=W;
X₄₂=Y, L, or F;
X₄₃=Q or L;
X₄₄=Q;
X₄₅=K or R;
X₄₆=P or Q;
X₄₇=G, D, or W;
X₄₈=Q, K, G, or T;
X₄₉=S, P, or T;
X₅₀=P or V;
X₅₁=K or Q;
X₅₂=L, R, or T;
X₅₃=L, R, or W;
X₅₄=V or I;
X₅₅=Y;
X₅₆=F, L, A, R, Y, or S;
X₅₇=A, V, or T;
X₅₈=S, P, or T;
X₅₉=T, E, K, N, D, or S;
X₆₀=R or L;
X₆₁=E, D, A, or H;
X₆₂=S or D;
X₆₃=G;
X₆₄=V or I;
X₆₅=P;
X₆₆=D, A, or S;
X₆₇=R;
X₆₈=F or L;
X₆₉=I, T, or S;
X₇₀=G;
X₇₁=S or R;
X₇₂=G;
X₇₃=S;
X₇₄=G or R;
X₇₅=T or Q;
X₇₆=D, Q, H, or S;
X₇₇=F or Y;
X₇₈=T or S;
X₇₉=L;
X₈₀=T, K, or N;
X₈₁=I;
X₈₂=S, H, or N;
X₈₃=S, R, P, or N;
X₈₄=V, A, L, or M;
X₈₅=Q or E;
X₈₆=A, E, S, or P;
X₈₇=E or D;
X₈₈=D;
X₈₉=L, V, A, F, or T;
X₉₀=A or G;
X₉₁=D, I, V, T, or S;
X₉₂=Y;
X₉₃=F or Y;
X₉₄=C;
X₉₅=Q, W, or L;
X₉₆=Q or H;
X₉₇=H, G, E, S, V, F, Y, or R;
X₉₈=Y, T, N, W, S, or G;
X₉₉=S, H, G, E, or K;
X₁₀₀=A, F, D, T, L, S, or Y;
X₁₀₁=P;
X₁₀₂=P, R, W, F, Y, or L;
X₁₀₃=T;
X₁₀₄=F;
X₁₀₅=G;
X₁₀₆=A, G, or S;
X₁₀₇=G;
X₁₀₈=T;
X₁₀₉=K;
X₁₁₀=L;
X₁₁₁=E;
X₁₁₂=I or L; X₁₁₃=K or is absent;
X₁₁₄=R or is absent.

### Example 5. Consensus Sequences of the CDR Sequences

The CDR sequences were analyzed further to identify consensus sequences of the heavy chain and light chain CDR sequences.

Consensus Sequence of the Heavy Chain CDRs of SEQ ID NOs: 75-98

The heavy chain CDRs of SEQ ID NOs: 75-98 were analyzed for variability across each amino acid position. SEQ ID NO: 153 represents a consensus sequence based on this analysis.

(SEQ ID NO: 153)
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂

wherein,
X₁=C;
X₂=A, S, or T, or is absent;
X₃=R, A, or S, or is absent;
X₄=G, S, D, E, R, H, or W, or is absent;
X₅=R, H, N, V, G, L, T, I, S, or D, or is absent;
X₆=D, Y, A, G, F, R, L, Q, V, T, or N, or is absent;
X₇=T, L, S, R, Y, or F, or is absent;
X₈=A, or S, or is absent;
X₉=S or is absent;
X₁₀=M or is absent;
X₁₁=T, I, R, or D, or is absent;
X₁₂=D, K, T, L, G, or V, or is absent;
X₁₃=G, N, T, L, F, or V, or is absent;
X₁₄=Y, W, V, L, or A, or is absent;
X₁₅=L, D, G, F, Y, R, V, E, or T, or is absent;
X₁₆=Y, N, S, H, R, G, K, F, or A, or is absent;
X₁₇=Y, H, D, W, A, P, N, or G, or is absent;
X₁₈=D, A, P, or R, or is absent;
X₁₉=F, M, or L;
X₂₀=D or A;
X₂₁=Y or V;
X₂₂=W.

Consensus Sequence of the Light Chain CDRs of SEQ ID NOs: 99-122
The light chains CDRs of SEQ ID NOs: 99-122 were analyzed for variability across each amino acid position. SEQ ID NO: 154 represents a consensus sequence based on this analysis.

X₁X₂X₃X₄X₅X₆X₇X₈X₉X₁₀X₁₁ (SEQ ID NO: 154)
wherein,
X₁=C;
X₂=H, W, M, L, W, or S;
X₃=Q or N;
X₄=Y, W, D, G, H, or S;
X₅=S, Y, F, N, H, L, G, or T;
X₆=K, S, I, R, E, or H;
X₇=L, F, Y, N, S, D, or V;
X₈=P or is absent;
X₉=Y, W, F, L, or R, or is absent;
X₁₀=T;
X₁₁=F.

Consensus sequence of the heavy chain CDRs of SEQ ID NOs: 123-135

The heavy chains CDRs of SEQ ID NOs: 123-135 were analyzed for variability across each amino acid position. SEQ ID NO: 155 represents a consensus sequence based on this analysis.

(SEQ ID NO: 155)
X₁X₂X₃X₄X₅X₆X₇X₈X₉X₁₀X₁₁X₁₂X₁₃X₁₄X₁₅X₁₆X₁₇X₁₈

wherein,
X₁=C;
X₂=A, T, or V;
X₃=R or is absent;
X₄=K, T, or D, or is absent;
X₅=R, H, L, or N, or is absent;
X₆=R, G, or Y, or is absent;
X₇=P, N, E, D, or A, or is absent;
X₈=L, S, R, T, G, N, or Y;
X₉=F, Y, W, or L;
X₁₀=Y, V, or F, or is absent;
X₁₁=Y, F, G, S, or L, or is absent;
X₁₂=A, N, S, G, R, or V, or is absent;
X₁₃=M, V, R, or L, or is absent;
X₁₄=D, Y, G, or T;
X₁₅=Y, S, or F;
X₁₆=A, D, or M, or is absent;
X₁₇=Y, V, or S, or is absent;
X₁₈=W or G.

Consensus sequence of the light chain CDRs of SEQ ID NOs: 136-148

The light chain CDRs of SEQ ID NOs: 136-148 were analyzed for variability across each amino acid position. SEQ ID NO: 156 represents a consensus sequence based on this analysis.

(SEQ ID NO: 156)
X₁X₂X₃X₄X₅X₆X₇X₈X₉X₁₀X₁₁X₁₂X₁₃X₁₄

wherein,
X₁=C;
X₂=Q, W, or L, or is absent;
X₃=Q;
X₄=G, E, Y, H, S, or R, or is absent;
X₅=T, S, G, or N, or is absent;
X₆=H or is absent;
X₇=V or F, or is absent;
X₈=Y or W, or is absent;
X₉=S, G, K, or E, or is absent;
X₁₀=A, T, L, S, D, or Y, or is absent;
X₁₁=P;
X₁₂=P, W, R, F, L, or Y;
X₁₃=T;
X₁₄=F.

Although various embodiments of the disclosure have been described and illustrated, it will be apparent to those skilled in the art in light of the present description that numerous modifications and variations can be made. The scope of the invention is defined more particularly in the appended claims.

What is claimed:

1. An antibody composition comprising a heavy chain having at least 80%, or at least 85%, or at least 90%, or at least 95% sequence identity with any one of SEQ ID NOs: 1-24.

2. The antibody composition of claim 1, wherein the heavy chain comprises any one of SEQ ID NOs: 1-24.

3. An antibody composition comprising a light chain having at least 80%, or at least 85%, or at least 90%, or at least 95% sequence identity with any one of SEQ ID NOs: 25-48.

4. The antibody composition of claim 3, wherein the light chain comprises any one of SEQ ID NOs: 25-48.

5. An antibody composition comprising a heavy chain having at least 80%, or at least 85%, or at least 90%, or at least 95% sequence identity with any one of SEQ ID NOs: 49-61.

6. The antibody composition of claim 5, wherein the heavy chain comprises any one of SEQ ID NOs: 49-61.

7. An antibody composition comprising a light chain having at least 80%, or at least 85%, or at least 90%, or at least 95% sequence identity with any one of SEQ ID NOs: 62-74.

8. The antibody composition of claim 7, wherein the light chain comprises any one of SEQ ID NOs: 62-74.

9. An antibody composition comprising:
(a) a heavy chain having at least 80%, or at least 85%, or at least 90%, or at least 95% sequence identity with any one of SEQ ID NOs: 1-24; and
(b) a light chain having at least 80%, or at least 85%, or at least 90%, or at least 95% sequence identity with any one of SEQ ID NOs: 25-48.

10. The antibody composition of claim 9, wherein:
(a) the heavy chain comprises any one of SEQ ID NOs: 1-24; and
(b) the light chain comprises any one of SEQ ID NOs: 25-48.

11. An antibody composition comprising:
(a) a heavy chain having at least 80%, or at least 85%, or at least 90%, or at least 95% sequence identity with any one of SEQ ID NOs: 49-61; and
(b) a light chain having at least 80%, or at least 85%, or at least 90%, or at least 95% sequence identity with any one of SEQ ID NOs: 62-74.

12. The antibody composition of claim 11, wherein:
(a) the heavy chain comprises any one of SEQ ID NOs: 49-61; and
(b) the light chain comprises any one of SEQ ID NOs: 62-74.

13. A method of treating an individual infected with SARS-CoV-2, the method comprising administering to the individual an effective amount of an antibody composition according to any one of claims 1-12.

14. A method of preventing SARS-CoV-2 infection in an individual, the method comprising administering to the individual an effective amount of an antibody composition according to any one of claims 1-12.

15. The method of claim 13 or 14, wherein the antibody composition is administered together with an adjuvant.

16. A method of identifying SARS-CoV-2 in an individual, the method comprising:
(a) isolating a biological sample from the individual;
(b) incubating the biological sample with an antibody composition according to any one of claims 1-12; and
(c) detecting a biological interaction between the biological sample and the antibody composition.

17. A kit for identifying SARS-CoV-2 in a biological sample, the kit comprising:
(a) an antibody composition according to any one of claims 1-12; and
(b) instructions of use.

18. An enzyme-linked immunosorbent assay (ELISA) test kit comprising:
(a) an antibody composition according to any one of claims 1-12; and
(b) instructions of use.

19. Use of an antibody composition according to any one of claims 1-12 for treating SARS-CoV-2.

20. Use of an antibody composition according to any one of claims 1-12 to formulate a medicament for treating SARS-CoV-2.


SEQUENCE LISTING (156 sequences)

SEQ ID NO: 1 - protein, 118 aa, Artificial Sequence
  Heavy Chain NP1_HC_A8
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCARGRDGYLYYFDYWGQGTTLTV

SEQ ID NO: 2 - protein, 115 aa, Artificial Sequence
  Heavy Chain NP1_HC_B2
EVQLQQSGAELVKPGASVKLSCTASGINIKDIYIHWVKQRPEQGLEWIGRIDPANGNIKYDPKFQDKATMTADTSSNIAYLQLSSLTSEDTAVYYCARWLQRFLDYWGQGTSVTV

SEQ ID NO: 3 - protein, 117 aa, Artificial Sequence
  Heavy Chain NP1_HC_B9
QVQLQQSGAELMKPGASVKISCKATGYTFSDYWIVWVKQRPGHGLEWIGEILPGSGNTNYNEKFKGKATFTADASSNTAYMELSSLSSEDSAVYYCASRLLLRRYFDVWGAGTPVTV

SEQ ID NO: 4 - protein, 114 aa, Artificial Sequence
  Heavy Chain NP1_HC_C4
QVQLQQSGPELVRPGVSVKISCKGSGYTFTDYVMHWVKQSRAKSLEWIGVISTYSGNTNYNQKFKGKATMTVDKSSSTAYMELARLTSEDSAIYYCARGTGDFDYWGQGTTLTV

SEQ ID NO: 5 - protein, 119 aa, Artificial Sequence
  Heavy Chain NP1_HC_C5
EVQLVESGGGLVKPGGSLKLSCAASGFIFSDYYMFWVRQTPEKRLEWVATISDGGRYTYYPDSVQGRFTISRHNAKNNLYLQMSSLKSEDTAIYYCARDRYDGWGSDFDVWGAGTTVTV

SEQ ID NO: 6 - protein, 115 aa, Artificial Sequence
  Heavy Chain NP1_HC_D3
QVQLQQSGPEVVRPGVSEKISCKGSGYTFTDYALHWVKQSHAKSLEWIGVISTYSGTTNYNQNFKGKATMAVDKSSRTAYMELARLTSDDSAIYYCASGGNYGLDYWGQGTSVTV

SEQ ID NO: 7 - protein, 118 aa, Artificial Sequence
  Heavy Chain NP1_HC_D4
EVQLVESGGDLVKPGGSLKLSCAASGFTFSDYYMSWVRQTPEKRLEWVATISDGGRYTYYPDSVKGRFTISRDNAKNNLYLQMGSLKSEDTAMYYCARGSTYGNPLDYWGQGTTLTVS

SEQ ID NO: 8 - protein, 117 aa, Artificial Sequence
  Heavy Chain NP1_HC_D6
QVTLKESGPGILQPSQTLSLTCSFSGFSLSTSGTGVSWIRQPSGKGLEWLAHIYWDDDKRYNPSLKSRLTLSKDTSTNQVFLKITSVDTADTATYYCARSNYGYDFDYWGPGTTLTV

SEQ ID NO: 9 - protein, 121 aa, Artificial Sequence
  Heavy Chain NP1_HC_D8
QVQLQQSGPELVKPGASVKMSCKASGYTFTDYVINWVKQRTGQGLEWIGEIYPESDNTFYNEKFRGRATLTADKSSNTAYMQLSSLTSGDSAVYFCARRGFYDGNYVRYFDVWGAGTTVTV

SEQ ID NO: 10 - protein, 118 aa, Artificial Sequence
  Heavy Chain NP1_HC_D9
QVQLQQSGPELVKPGASVKISCKASGYSFTGYFMNWVKQSHGRSLEWIGRINSYNGDTFYNQKFKGKATLTVDKSSSTAHMELLSLTSEDSAVYYCGLTTVVATKYFDYWGQGTTLTV

SEQ ID NO: 11 - protein, 122 aa, Artificial Sequence
  Heavy Chain NP1_HC_E2
QVQLQQPGAELVKPGAPVKLSCKASGYTFTSYWMNWVKQRPGRDLEWIGRIDPSDSETHYNQKFKDKATLTVDKSSSTAYIQLSSLTSEDSAVYYCSREVATATKGVFSWFAYWGQGTLVTV

SEQ ID NO: 12 - protein, 119 aa, Artificial Sequence
  Heavy Chain NP1_HC_E4
QVQLKESGPGLVAPSQSLSITCTVSGFSLTSYGVHWVRQPPGKGLEWLVVIWSDGSTTYNSALKSRLSISKDNSKSQVFLKMNSLQTDDTAMYYCARNYYGSSYYAMDYWGQGTSVTVS

SEQ ID NO: 13 - protein, 119 aa, Artificial Sequence
  Heavy Chain NP1_HC_E6
QVQLQQSGPELVKPGASVKISCKASGYSFTAYFMNWVKQSHGKSLEWIGRINPYNGDTFYNQKFKGKATLTVDKSSSTAHMDLLSLTSEDSAVYYCGLTTLVATKYFDYWGQGTTLTVS

SEQ ID NO: 14 - protein, 116 aa, Artificial Sequence
  Heavy Chain NP1_HC_E7
EVQLQQSGAELVKPGASVKLSCTASGINIKDIYIHWVKQRPEQGLEWIGRIDPANGNIKYDPKFQDKATMTADTSSNIAYLQLSSLTSEDTAVYYCARWLQRFLDYWGQGTSVTVS

SEQ ID NO: 15 - protein, 116 aa, Artificial Sequence
  Heavy Chain NP1_HC_E9
QVQLQQSGPELVKPGTLVKISCKASGYTFTSYDINWVKQRPGQGLEWIGWIYPGDGSTKYNEKFKGKATLTADTSSSTAYMQLNSLTSENSAVYFCARGLVGAMDYWGQGTSVTVS

SEQ ID NO: 16 - protein, 118 aa, Artificial Sequence
  Heavy Chain NP1_HC_E12
QIQLVQSGPELKKPGETVKISCKASGYTFTDYSIHWVKQAPGKGLKWMGWINTYTGEPTYADDFKGRFAFSLETSATTAYLQINNLKNEDSATYFCARSHDNYDNHFDYWGQGTTLTV

SEQ ID NO: 17 - protein, 122 aa, Artificial Sequence
  Heavy Chain NP1_HC_F3
EVQLVESGGGLVQPGGSLKLSCAASGFTFSSYTMSWVRQTPEKRLEWVAFISYSGTNVYYPDTVKGRFTISRDVAKNALYLQMTSLKSEDTALYFCARHGRFTTVVEGYFDVWGAGTTVTVS

SEQ ID NO: 18 - protein, 117 aa, Artificial Sequence
  Heavy Chain NP1_HC_F8
QVQLQQSGPELVRPGVSVKISCKGSGYTFTDYAMHWVKQSHAKSLEWIGVISTYYGNTNYNQKFKGKATMTVDKSSSTAYMELARLTSEDSAIYYCARGITTAPFDYWGQGTTLTVS

SEQ ID NO: 19 - protein, 117 aa, Artificial Sequence
  Heavy Chain NP1_HC_F9
EVQLVESGGGLVRPGGSLKLSCAASGFTFSDYYIYWVRQTPEKRLEWVATISDGGRYTYYPDSVKGRFTISRDNAKNNLYLQMTSLKSEDTAMYYCTRGDDYGGRLAYWGQGTLVTV

SEQ ID NO: 20 - protein, 113 aa, Artificial Sequence
  Heavy Chain NP1_HC_F11
QVQLQQSGAELVRPGASVKISCKTFGYSFTYHHINWVKQRPGQGLDWIGYINPYNDYSGYDHNFKGKATLTVDKSSSTAYMELSSLTSEDSAIYYCTAERGSYAMDYWGQGTS

SEQ ID NO: 21 - protein, 117 aa, Artificial Sequence
  Heavy Chain NP1_HC_G4
DVQLQESGPGLVKPSQSLSLTCTVTGYSITSDSAWNWIRQFPRNKLEWMGYISYSGATTYNPSLKSRFSITRDTSKNQFFLQLNSVTTEDTATYYCAREGIHYAMDYWGQGTSVTVS

SEQ ID NO: 22 - protein, 125 aa, Artificial Sequence
  Heavy Chain NP1_HC_G7
EVQLQQSGAELVRPGALVKLSCKASGFDIKDFYMNWVRQRPEQGLEWIGWIDPENGNIKYNPKFQGKANITAGTSSNTAYLQLSSLTSEDTAVYYCAREGLSSMITTVYYYAMDYWGQGTSVTVS

SEQ ID NO: 23 - protein, 119 aa, Artificial Sequence
  Heavy Chain NP1_HC_H6
DVQLQESGPGLVKPSQSLSLTCTVTGYSSTSDYAWNWIRQFPGNKLEWMGYVSYSGSTSYNPSLKSRNSITRDTSKNQFFLQLNSVTTEDTATYYCARGRDGYLYYFDYWGQGTTLTVS

SEQ ID NO: 24 - protein, 118 aa, Artificial Sequence
  Heavy Chain NP1_HC_H8
QVQLQQSGAELVKPGASVKISCKATGYTLSSDWIEWVKQRPGHGLEWIGEILPGSDNTDYNEKFTGKATFTADTSSNTAYMQLSSLTSEDSAVYYCARRKFLRRYFDVWGAGTTVTVS

SEQ ID NO: 25 - protein, 104 aa, Artificial Sequence
  Light Chain NP1_kLC_A8
DIQMTQTTSSLSASLEDRVTISCSASQGIRNYLNWYQQKPDGTVKLLIFFTSTLHSGVPSRFSGSGSGTDYSLTISNLEPEDIATYYCHQYSKLPYTFGGGTKL

SEQ ID NO: 26 - protein, 109 aa, Artificial Sequence
  Light Chain NP1_kLC_B2
DIVMTQAAPSIPVTPGESVSISCRSNQSLLHSSGNTFLYWFLQRPGQSPQLLIYRMSNLASGVPDRFSGSGSGTAFTLRISRVEAEDVGVYYCMQHLEYPLTFGGGTKL

SEQ ID NO: 27 - protein, 108 aa, Artificial Sequence
  Light Chain NP1_kLC_B9
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPFTFGGGTKL

SEQ ID NO: 28 - protein, 115 aa, Artificial Sequence
  Light Chain NP1_kLC_C4
DIVMTQSPSSLAVSVGEKVTMSCKSGQSLLYSSNQKNYLAWYQQKPGQSPKLLIYWASTRESGVPDRFTGSGSGTDFTLTISSVKAEDLAVYFCQQYYSYPWTFGGGTKLEIKRT

SEQ ID NO: 29 - protein, 105 aa, Artificial Sequence
  Light Chain NP1_kLC_C5
ENVLTQSPAIMSASLGERVTMTCTASSSVNSNYLHWYQQKPGSSPKLWIYSTSNLASGVPTRFSGSGSGTSYSLTISSMEAEDAATYYCHQYHRSPRTFGGGTKL

SEQ ID NO: 30 - protein, 115 aa, Artificial Sequence
  Light Chain NP1_kLC_D3
DIVMTQSPSSLSMSAGEKVTMSCKSSQSLLNSGNQKNYLAWYQQKPGQPPKLLIYGASTRESGVPDRFTGSGSGTDFTLTISSVQAEDLAVYYCQNDHSYPYTFGWGTKLELKRT

SEQ ID NO: 31 - protein, 109 aa, Artificial Sequence
  Light Chain NP1_kLC_D4
DIQMTQTTSSLSASRGDRVTISCSASQDISNFLNWYQQKPDGTVKLLIYYTSSLHSGVPSRFSGSGSGTDYSLTISNLEPEDIATYYCQQYSKLPFTFGSGTKLELKRT

SEQ ID NO: 32 - protein, 108 aa, Artificial Sequence
  Light Chain NP1_kLC_D6
DIVMTQSQKFMSTSVGDRVSVTCKASQNVGTNVAWYQQKPGQSPKALIYSASYRYSGVPDRFTGSGSGTDFTLTISNMQSEDLAEYFCQQYNSYPTFGGGTKLELKRT

SEQ ID NO: 33 - protein, 109 aa, Artificial Sequence
  Light Chain NP1_kLC_D8
DIQMTQTTSSMYASLGERVTITCKASQDIKSYLSWYQQKPWKSPKTLIYYATSLADGVPSRFSGSGSGQDYSLTISSLESDDTATYYCLQHGESPLTFGAGTKLELKRT

SEQ ID NO: 34 - protein, 115 aa, Artificial Sequence
  Light Chain NP1_kLC_D9
DIVMTQSPSSLTVSVGEKVTMSCKSSQSLLYSSNQKNFLAWYQQKPGQSPKLLIYWASTRESGVPDRFTGSGSGTDFTLTISSVKAEDLAVYYCQQYYSYPLTFGAGTKLELKRT

SEQ ID NO: 35 - protein, 109 aa, Artificial Sequence
  Light Chain NP1_kLC _E2
DIQMTQTTSSLSASLGDRVTISCSASQDIRDYLNWYQQKPDGTVKLLIYYTSILHSGVPSRFSGSGSGTDYSLTISNLASEDIATYYCQQYSKLPWTFGGGTKLEIKRT

SEQ ID NO: 36 - protein, 109 aa, Artificial Sequence
  Light Chain NP1_kLC_E4
DIKMTQSPSSMYASLGERVTITCKASQDIKSYLSWYQQKPWKSPKTLIYYATSLADGVPSRFSGSGSGQDYSLTISSLESDDTATYYCLQHGESPFTFGSGTKLELKRT

SEQ ID NO: 37 - protein, 115 aa, Artificial Sequence
  Light Chain NP1_kLC_E6
DIVMTQSPSSLAVSVGEKVTMSCKSSQSLLYSSNQKNFLAWYQQKPGQSPKLLIYWASTRESGVPDRFTGSGSGTDFTLTISSVKAEDLAVYYCQQYFSYPLTFGAGTKLELKRT

SEQ ID NO: 38 - protein, 113 aa, Artificial Sequence
  Light Chain NP1_kLC_E7
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHLWTFGGGTKLEIKRT

SEQ ID NO: 39 - protein, 109 aa, Artificial Sequence
  Light Chain NP1_kLC_E9
DIVMTQSQKFMSTSVGDRVSVTCKASQNVLNNVAWYQQKPGQSPKALIYSASYRYSGVPDRFTGSGSGTDFTLTISNVQSEDLAEYFCQQYNSYPLTFGDGTKLELKRT

SEQ ID NO: 40 - protein, 108 aa, Artificial Sequence
  Light Chain NP1_kLC_E12
ENVLTQSPALMSASPGGKVTMTCSASSSVSYMYWYQQKPRSSPKPWIYLTSNLASGVPARFSGSGSGTSYSLTISSMEAEDAATYYCQQWSINPYTFGGGTKLEIKRT

SEQ ID NO: 41 - protein, 109 aa, Artificial Sequence
  Light Chain NP1_kLC_F3
DIVMTQSPATLSVTPGDRVSLSCRASQSISDYLHWYQQKSHESPRLLIKYVSQSISGIPSRFSGSGSGSDFTLSINSVEPEDVGIYYCQNGHSFPWTFGGGTKLEIKRT

SEQ ID NO: 42 - protein, 109 aa, Artificial Sequence
  Light Chain NP1_kLC_F8
DIVMTQSHKFMSTSVGDRVSITCKASQDVSTAVAWYQQKPGQSPKLLIYWESTRHTGVPDRFTGSGSGTDLTLTISNVQSEDLADYFCQQYSSYPYTFGGGTKLEIKRT

SEQ ID NO: 43 - protein, 109 aa, Artificial Sequence
  Light Chain NP1_kLC_F9
DIQMTQTTSSQSASLGDRVTINCSASQGISNYLNWYQQKPDGTVKLLIYYTSSLHSGVPSRFSGSGSGTEYSLTISNLEPEDIATYYCQQYSKLPYTFGGGTKLEIKRT

SEQ ID NO: 44 - protein, 114 aa, Artificial Sequence
  Light Chain NP1_kLC_F11
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPFTFGSGTKLEIKRT

SEQ ID NO: 45 - protein, 109 aa, Artificial Sequence
  Light Chain NP1_kLC_G4
DIKMTQSPSSMYASLGERVTITCKASQDIKSYLSWYQQKPWKSPKTLIYYATSLADGVPSRFSGSGSGQDYSLTISSLESDDTATYYCLQHGESPLTFGAGTKLEIKRT

SEQ ID NO: 46 - protein, 114 aa, Artificial Sequence
  Light Chain NP1_kLC_G7
DIVMTQAAASIPVSPGESVSISCRSNQSLLHSSGITFLYWFLQRPGQSPHLLIYRMSNLASGVPNRFSGSGSGTAFTLRISKVEAEDVGVYYCMQHLEYPLTFGGGTKLEIKRT

SEQ ID NO: 47 - protein, 109 aa, Artificial Sequence
  Light Chain NP1_kLC_H6
DIQMTQTTSSLSASLGDRVTISCSASQDINNNLNWYQQKPDVTVKLLIYYTSSLHSGVPSRLSGSGSGTDYSLTISNLEPEDIATYYCQQYSKFPYTFGGGTKLEIKRT

SEQ ID NO: 48 - protein, 109 aa, Artificial Sequence
  Light Chain NP1_kLC_H8
DIQMTQTTSSLSASLGDRVTISCSASQDINNNLNWYQQKPDGTVKLLIYYTSSLHSGVPSRLSGSGSGTDYSLTISNLEPEDIATYYCQQYSKFPYTFGGGTKLEIKRT

SEQ ID NO: 49 - protein, 117 aa, Artificial Sequence
  Heavy Chain NP2_HC_A10
EVQLQESGAELVRPGSSVKISCKVSGYTFSNYWMNWVKQRPGQGLEWIGQIYPGDDSTYYNGKFKDKAKMTADKSSSTAYIQLSSLTSEDSAVYFCARRPLFYAMDYWGQGTSVTVS

SEQ ID NO: 50 - protein, 119 aa, Artificial Sequence
  Heavy Chain NP2_HC_A11
EVQLQESGPELKKPGETVKISCKASGYTFTNYGMNWVKQAPGKGLKWMGWINTYTGEPTYADDFKGRFAFSLDTSTTTANLQINNLTNDDMATYFCARTRETWGNVDSAYWGQGTLVTV

SEQ ID NO: 51 - protein, 120 aa, Artificial Sequence
  Heavy Chain NP2_HC_B1
EVQLQESGPERKKPGETVKISCKASGYSLTYHSMHWVKQAPGKGLKWMGWINTETGEPTYADDFKGRFAFSLETSASTAYLQINNLKNEDTATYFCTRKHGNSYYYAMDYWGQGTSVTVS

SEQ ID NO: 52 - protein, 119 aa, Artificial Sequence; identical to SEQ ID NO: 50
  Heavy Chain NP2_HC-C1

SEQ ID NO: 53 - protein, 120 aa, Artificial Sequence
  Heavy Chain NP2_HC_C12
EVQLQESGGGLVRPGGSLKLSCAASGFTFSDYYMYWVRQTPEKRLEWVATISDGGRYSYYPDSVKGRFTVSRDNAKNNLYLQMSSLKSEDTAMYYCVRDRYDGWGSDFDVWGAGTTVTVS

SEQ ID NO: 54 - protein, 117 aa, Artificial Sequence
  Heavy Chain NP2_HC_D1
EVQLQESGPELVKPGASVKMSCKASGYTFTRYVMHWVKQKPGQGLEWIGYINPYNDGTKYNEKFKGKATLTSDKSSSTAYMELRSLTSEDSAVYYCARLGPRYYFDYWGQGTTLTVS

SEQ ID NO: 55 - protein, 118 aa, Artificial Sequence
  Heavy Chain NP2_HC_D8
EVQLQESGAELMKPGASVKISCKATGSTFSSYWIEWVKQRPGHGLEWIGEILPGSGNTNYNEKFKAKATFTADTASNTAYMQLTSLTSEDSAVHYCARRWFLRRYFDVWGAGTTVTVS

SEQ ID NO: 56 - protein, 120 aa, Artificial Sequence
  Heavy Chain NP2_HC_D12
EVQLQESGGGLVKPGGSLKLSCAASGFTFSDYYMYWVRQTPEKRLEWVATISDGGRYTYYPDSVQGRFTISRDNAKNNLYLQMSSLKSEDTAMYYCARDRYDGWGSDFDVWGAGTTVTVS

SEQ ID NO: 57 - protein, 120 aa, Artificial Sequence
  Heavy Chain NP2_HC_E3
EVQLQESGGGLVKPGGSLKLSCAASGFTFSDYYMYWVRQTPEKRLEWVATISDGGRYTYYPDSVQGRFTISRDNAKNNLYLQMSSLKSEDTAMYYCARDRYDGLGSDFDVWGAGTTVTVS

SEQ ID NO: 58 - protein, 121 aa, Artificial Sequence
  Heavy Chain NP2_HC_E4
EVQLQESGGDSVKPGGSLKLSCAASGFTFSTYTMSWVRQTPEKRLEWVATISSGGTYTYYPDSVKGRFTISRDNAKSTLYLQMSSLKSEDTALYHCTRDRDYYVSSRGFAYWGQGTLVTVS

SEQ ID NO: 59 - protein, 118 aa, Artificial Sequence
  Heavy Chain NP2_HC_F6
EVQLQESGTELMKPGASVKISCKATGSTFSSYWIEWVKQRPEHGLEWIGEILPGSGNTNYNEKFRAKATFTADTASNTAYMQLSSLTSEDSAVHYCARRWFLRRYFDVWGAGTTVTVS

SEQ ID NO: 60 - protein, 119 aa, Artificial Sequence
  Heavy Chain NP2_HC_H1
EVQLQESGGGLVQPGRSLKLSCAASGFTFSDYYLFWFRQTPEKRLEWVATISDGGRYTYYLDSVKGRFTISRDNAKNKVYLQMSSLKSEDTAMYYCARDNANYGGYFDYWGQGTTLTVS

SEQ ID NO: 61 - protein, 120 aa, Artificial Sequence
  Heavy Chain NP2_HC-H4
EVQLQESGGGLVKPGGSLKLSCAASGFIFSDYYMFWVRQTPEKRLEWVATISDGGRYTYYPDSVQGRFTISRDNAKNNLYLQTSSLKSEDTAIYYCARDRYDGWGSDFDVWGAGTTVTVS

SEQ ID NO: 62 - protein, 114 aa, Artificial Sequence
  Light Chain NP2_kLC_A10
DIQMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSAPPTFGAGTKLEIKR

SEQ ID NO: 63 - protein, 108 aa, Artificial Sequence
  Light Chain NP2_kLC_A11
DIQMTQSPSSLSVSVGETVTITCRASENVYSNLAWYQQKQGKSPQLRVYAATDLADGVPSRFSGRGSGTQYSLKINNLQSEDFGTYYCQHVWGTPWTFGGGTKLEIKR

SEQ ID NO: 64 - protein, 108 aa, Artificial Sequence
  Light Chain NP2_kLC_B1
DIQMTQSPSSLSVSLGDRVTISCSVSQDISDYLNWYQQKPDGTVKLLIYFTSSLHSGVPSRFSGSGSGTDYSLTISNLEPEDLATYYCQQYSKLPFTFGSGTKLEIKR

SEQ ID NO: 65 - protein, 113 aa, Artificial Sequence
  Light Chain NP2_kLC_C1
DVVMTQTPLTLSVTIGQPASISCKSSQTLLDSDGKTYLNWLLQRPGQSPKRLIYLVSELDSGVPDRFTGSGSGTDFTLKISRVEAEDLGIYYCWQGTHFPRTFGGGTKLEIKR

SEQ ID NO: 66 - protein, 108 aa, Artificial Sequence
  Light Chain NP2_kLC_C12
DVQITQSPSSMYASLGERVTITCKASQDIKSYLSWYQQKPWKSPKTLIYYATSLADGVPSRFSGSGSGQDYSLTISSLESDDTATYYCLQHGESPWTFGGGTKLEIKR

SEQ ID NO: 67 - protein, 108 aa, Artificial Sequence
  Light Chain NP2_kLC_D1
DVQITQSPSSLSVSVGETVTITCRASENIYSNLAWYQQKQGKSPQLLVYAATNLADGVPSRFSGSGSGTHYSLKINNLQSEDFGSYYCQHFWGTPWTFGGGTKLEIKR

SEQ ID NO: 68 - protein, 112 aa, Artificial Sequence
  Light Chain NP2_kLC_D8
ENVLTQSPASLAVSLGQKATISCKASQSVDYDGESYMNWYQQKPGQPPKLLIYAASNLESGIPARFSGSGSGTDFTLNIHPAEEEDVATYYCQQSNGDPFTFGGGTKLEIKR

SEQ ID NO: 69 - protein, 112 aa, Artificial Sequence
  Light Chain NP2_kLC_D12
ENVLTQSPASLAVSLGQRATISCRASESVGSYGNSFMHWYQQKPGQPPKLLIYRAPNLESGIPARFSGSGSRTDFTLTINPVEADDVATYYCQQSNEDPYTFGGGTKLELKR

SEQ ID NO: 70 - protein, 108 aa, Artificial Sequence
  Light Chain NP2_kLC_E3
DVQITQSPSSMYASLGERVTITCKASQDIKSYLSWYQQKPWKSPKTLIYYATSLADGVPSRLSGSGSGQDYSLTISSLESDDTATYYCLQHGESPLTFGAGTKLEIKR

SEQ ID NO: 71 - protein, 113 aa, Artificial Sequence
  Light Chain NP2_kLC_E4
DVVMTQTPLTLSVTIGQPASISCKSSQSLLDSDGKTHLNWLLQRPGQSPKRLIYLVSKLDSGVPDRFTGSGSGTDFTLKISRVEAEDLGVYYCWQETHFPWTFGGGTKLELKR

SEQ ID NO: 72 - protein, 112 aa, Artificial Sequence
  Light Chain NP2_kLC_F6
DVQITQSPASLAVSLGQRATISCKASQSVDYDGDSYMNWYQQKPGQPPKLLIYAASNLESGIPARFSGSGSGTDFTLNIHPVEEEDAATYYCQQSNEDPFTFGGGTKLEIKR

SEQ ID NO: 73 - protein, 108 aa, Artificial Sequence
  Light Chain NP2_kLC_H1
DIQMTQSPSSMYASLGERVTITCKASQDIKSYLSWYQQKPWKSPKTLIYYATSLADGVPSRFSGSGSGQDYSLTISSLESDDTATYYCLQHGESPFTFGSGTKLEIKR

SEQ ID NO: 74 - protein, 105 aa, Artificial Sequence
  Light Chain NP2_kLC_H4
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLEL

SEQ ID NO: 75 - protein, 15 aa, Artificial Sequence
  NP1_HC_A8 CDR sequence within SEQ ID NO 1
CARGRDGYLYYFDYW

SEQ ID NO: 76 - protein, 12 aa, Artificial Sequence
  NP1_HC_B2 CDR sequence within SEQ ID NO 2
CARWLQRFLDYW

SEQ ID NO: 77 - protein, 14 aa, Artificial Sequence
  NP1_HC_B9 CDR sequence within SEQ ID NO 3
CASRLLLRRYFDVW

SEQ ID NO: 78 - protein, 11 aa, Artificial Sequence [sequence omitted]

SEQ ID NO: 79 - protein, 16 aa, Artificial Sequence
  NP1_HC_C5 CDR sequence within SEQ ID NO 5
CARDRYDGWGSDFDVW

SEQ ID NO: 80 - protein, 12 aa, Artificial Sequence
  NP1_HC_D3 CDR sequence within SEQ ID NO 6
CASGGNYGLDYW

SEQ ID NO: 81 - protein, 14 aa, Artificial Sequence
  NP1_HC_D4 CDR sequence within SEQ ID NO 7
CARGSTYGNPLDYW

SEQ ID NO: 82 - protein, 13 aa, Artificial Sequence
  NP1_HC_D6 CDR sequence within SEQ ID NO 8
CARSNYGYDFDYW

SEQ ID NO: 83 - protein, 18 aa, Artificial Sequence
  NP1_HC_D8 CDR sequence within SEQ ID NO 9
CARRGFYDGNYVRYFDVW

SEQ ID NO: 84 - protein, 15 aa, Artificial Sequence
  NP1_HC_D9 CDR sequence within SEQ ID NO 10
CGLTTVVATKYFDYW

SEQ ID NO: 85 - protein, 19 aa, Artificial Sequence
  NP1_HC_E2 CDR sequence within SEQ ID NO 11
CSREVATATKGVFSWFAYW

SEQ ID NO: 86 - protein, 16 aa, Artificial Sequence
  NP1_HC_E4 CDR sequence within SEQ ID NO 12
CARNYYGSSYYAMDYW

SEQ ID NO: 87 - protein, 15 aa, Artificial Sequence
  NP1_HC_E6 CDR sequence within SEQ ID NO 13
CGLTTLVATKYFDYW

SEQ ID NO: 88 - protein, 12 aa, Artificial Sequence
  NP1_HC_E7 CDR sequence within SEQ ID NO 14
CARWLQRFLDYW

SEQ ID NO: 89 - protein, 12 aa, Artificial Sequence
  NP1_HC_E9 CDR sequence within SEQ ID NO 15
CARGLVGAMDYW

SEQ ID NO: 90 - protein, 15 aa, Artificial Sequence
  NP1_HC_E12 CDR sequence within SEQ ID NO 16
CARSHDNYDNHFDYW

SEQ ID NO: 91 - protein, 18 aa, Artificial Sequence
  NP1_HC_F3 CDR sequence within SEQ ID NO 17
CARHGRFTTVVEGYFDVW

SEQ ID NO: 92 - protein, 13 aa, Artificial Sequence
  NP1_HC_F8 CDR sequence within SEQ ID NO 18
CARGITTAPFDYW

SEQ ID NO: 93 - protein, 14 aa, Artificial Sequence
  NP1_HC_F9 CDR sequence within SEQ ID NO 19
CTRGDDYGGRLAYW

SEQ ID NO: 94 - protein, 13 aa, Artificial Sequence
  NP1_HC_F11 CDR sequence within SEQ ID NO 20
CTAERGSYAMDYW

SEQ ID NO: 95 - protein, 13 aa, Artificial Sequence
  NP1_HC_G4 CDR sequence within SEQ ID NO 21
CAREGIHYAMDYW

SEQ ID NO: 96 - protein, 21 aa, Artificial Sequence
  NP1_HC_G7 CDR sequence within SEQ ID NO 22
CAREGLSSMITTVYYYAMDYW

SEQ ID NO: 97 - protein, 15 aa, Artificial Sequence
  NP1_HC_H6 CDR sequence within SEQ ID NO 23
CARGRDGYLYYFDYW

SEQ ID NO: 98 - protein, 14 aa, Artificial Sequence
  NP1_HC_H8 CDR sequence within SEQ ID NO 24
CARRKFLRRYFDVW

SEQ ID NO: 99 - protein, 11 aa, Artificial Sequence
  NP1_kLC_A8 CDR sequence within SEQ ID NO 25
CHQYSKLPYTF

SEQ ID NO: 100 - protein, 11 aa, Artificial Sequence
  NP1_kLC_B2 CDR sequence within SEQ ID NO 26
CMQHLEYPLTF

SEQ ID NO: 101 - protein, 11 aa, Artificial Sequence
  NP1_kLC_B9 CDR sequence within SEQ ID NO 27
CQQSNEDPFTF

SEQ ID NO: 102 - protein, 11 aa, Artificial Sequence [sequence omitted]

SEQ ID NO: 103 - protein, 11 aa, Artificial Sequence
  NP1_kLC_C5 CDR sequence within SEQ ID NO 29
CHQYHRSPRTF

SEQ ID NO: 104 - protein, 11 aa, Artificial Sequence
  NP1_kLC_D3 CDR sequence within SEQ ID NO 30
CQNDHSYPYTF

SEQ ID NO: 105 - protein, 10 aa, Artificial Sequence
  NP1_kLC_D4 CDR sequence within SEQ ID NO 31
QQYSKLPFTF

SEQ ID NO: 106 - protein, 10 aa, Artificial Sequence [sequence omitted]

SEQ ID NO: 107 - protein, 11 aa, Artificial Sequence
  NP1_kLC_D8  CDR sequence within SEQ ID NO 33
CLQHGESPLTF

SEQ ID NO: 108 - protein, 11 aa, Artificial Sequence [sequence omitted]

SEQ ID NO: 109 - protein, 11 aa, Artificial Sequence
  NP1_kLC _E2 CDR sequence within SEQ ID NO 35
CQQYSKLPWTF

SEQ ID NO: 110 - protein, 11 aa, Artificial Sequence
  NP1_kLC_E4 CDR sequence within SEQ ID NO 36
CLQHGESPFTF

SEQ ID NO: 111 - protein, 11 aa, Artificial Sequence [sequence omitted]

SEQ ID NO: 112 - protein, 10 aa, Artificial Sequence
  NP1_kLC_E7 CDR sequence within SEQ ID NO 38
CWQGTHLWTF

SEQ ID NO: 113 - protein, 11 aa, Artificial Sequence [sequence omitted]

SEQ ID NO: 114 - protein, 11 aa, Artificial Sequence
  NP1_kLC_E12 CDR sequence within SEQ ID NO 40
CQQWSINPYTF

SEQ ID NO: 115 - protein, 11 aa, Artificial Sequence
  NP1_kLC_F3 CDR sequence within SEQ ID NO 41
CQNGHSFPWTF

SEQ ID NO: 116 - protein, 11 aa, Artificial Sequence [sequence omitted]

SEQ ID NO: 117 - protein, 11 aa, Artificial Sequence
  NP1_kLC_F9 CDR sequence within SEQ ID NO 43
CQQYSKLPYTF

SEQ ID NO: 118 - protein, 11 aa, Artificial Sequence
  NP1_kLC_F11 CDR sequence within SEQ ID NO 44
CSQSTHVPFTF

SEQ ID NO: 119 - protein, 11 aa, Artificial Sequence
  NP1_kLC_G4 CDR sequence within SEQ ID NO 45
CLQHGESPLTF

SEQ ID NO: 120 - protein, 11 aa, Artificial Sequence
  NP1_kLC_G7 CDR sequence within SEQ ID NO 46
CMQHLEYPLTF

SEQ ID NO: 121 - protein, 11 aa, Artificial Sequence
  NP1_kLC_H6 CDR sequence within SEQ ID NO 47
CQQYSKFPYTF

SEQ ID NO: 122 - protein, 11 aa, Artificial Sequence
  NP1_kLC_H8 CDR sequence within SEQ ID NO 48
CQQYSKFPYTF

SEQ ID NO: 123 - protein, 13 aa, Artificial Sequence
  NP2_HC_A10 CDR sequence within SEQ ID NO 49
CARRPLFYAMDYW

SEQ ID NO: 124 - protein, 16 aa, Artificial Sequence
  NP2_HC_A11 CDR sequence within SEQ ID NO 50
CARTRETWGNVDSAYW

SEQ ID NO: 125 - protein, 16 aa, Artificial Sequence
  NP2_HC_B1 CDR sequence within SEQ ID NO 51
CTRKHGNSYYYAMDYW

SEQ ID NO: 126 - protein, 16 aa, Artificial Sequence
  NP2_HC-C1 CDR sequence within SEQ ID NO 52
CARTRETWGNVDSAYW

SEQ ID NO: 127 - protein, 16 aa, Artificial Sequence
  NP2_HC_C12 CDR sequence within SEQ ID NO 53
CVRDRYDGWGSDFDVW

SEQ ID NO: 128 - protein, 13 aa, Artificial Sequence
  NP2_HC_D1 CDR sequence within SEQ ID NO 54
CARLGPRYYFDYW

SEQ ID NO: 129 - protein, 14 aa, Artificial Sequence
  NP2_HC_D8 CDR sequence within SEQ ID NO 55
CARRWFLRRYFDVW

SEQ ID NO: 130 - protein, 16 aa, Artificial Sequence
  NP2_HC_D12 CDR sequence within SEQ ID NO 56
CARDRYDGWGSDFDVW

SEQ ID NO: 131 - protein, 17 aa, Artificial Sequence
  NP2_HC_E3 CDR sequence within SEQ ID NO 57
CARDRYDGLGVLTSMSG

SEQ ID NO: 132 - protein, 17 aa, Artificial Sequence
  NP2_HC_E4 CDR sequence within SEQ ID NO 58
CTRDRDYYVSSRGFAYW

SEQ ID NO: 133 - protein, 14 aa, Artificial Sequence
  NP2_HC_F6 CDR sequence within SEQ ID NO 59
CARRWFLRRYFDVW

SEQ ID NO: 134 - protein, 15 aa, Artificial Sequence
  NP2_HC_H1 CDR sequence within SEQ ID NO 60
CARDNANYGGYFDYW

SEQ ID NO: 135 - protein, 16 aa, Artificial Sequence
  NP2_HC-H4 CDR sequence within SEQ ID NO 61
CARDRYDGWGSDFDVW

SEQ ID NO: 136 - protein, 11 aa, Artificial Sequence [sequence omitted]

SEQ ID NO: 137 - protein, 11 aa, Artificial Sequence
  NP2_kLC_A11 CDR sequence within SEQ ID NO 63
CQHVWGTPWTF

SEQ ID NO: 138 - protein, 11 aa, Artificial Sequence
  NP2_kLC_B1 CDR sequence within SEQ ID NO 64
CQQYSKLPFTF

SEQ ID NO: 139 - protein, 11 aa, Artificial Sequence
  NP2_kLC_C1 CDR sequence within SEQ ID NO 65
CWQGTHFPRTF

SEQ ID NO: 140 - protein, 11 aa, Artificial Sequence
  NP2_kLC_C12 CDR sequence within SEQ ID NO 66
CLQHGESPWTF

SEQ ID NO: 141 - protein, 11 aa, Artificial Sequence
  NP2_kLC_D1 CDR sequence within SEQ ID NO 67
CQHFWGTPWTF

SEQ ID NO: 142 - protein, 11 aa, Artificial Sequence
  NP2_kLC_D8 CDR sequence within SEQ ID NO 68
CQQSNGDPFTF

SEQ ID NO: 143 - protein, 11 aa, Artificial Sequence
  NP2_kLC_D12 CDR sequence within SEQ ID NO 69
CQQSNEDPYTF

SEQ ID NO: 144 - protein, 11 aa, Artificial Sequence
  NP2_kLC_E3 CDR sequence within SEQ ID NO 70
CLQHGESPLTF

SEQ ID NO: 145 - protein, 11 aa, Artificial Sequence
  NP2_kLC_E4 CDR sequence within SEQ ID NO 71
CWQETHFPWTF

SEQ ID NO: 146 - protein, 11 aa, Artificial Sequence
  NP2_kLC_F6 CDR sequence within SEQ ID NO 72
CQQSNEDPFTF

SEQ ID NO: 147 - protein, 11 aa, Artificial Sequence
  NP2_kLC_H1 CDR sequence within SEQ ID NO 73
CLQHGESPFTF

SEQ ID NO: 148 - protein, 11 aa, Artificial Sequence
  NP2_kLC_H4 CDR sequence within SEQ ID NO 74
CQQRSSYPLTF

SEQ ID NO: 149 - protein, 129 aa, Artificial Sequence
  Consensus Sequence of the Heavy Chain Sequences of SEQ ID NOs 1-24
  misc_feature (1)..(1): Xaa = D, Q, or E
  misc_feature (2)..(2): Xaa = V or I
  misc_feature (3)..(3): Xaa = Q or T
  misc_feature (5)..(5): Xaa = Q, K, or V
  misc_feature (7)..(7): Xaa = S or P
  misc_feature (9)..(9): Xaa = P, G, or A
  misc_feature (10)..(10): Xaa = G, D, or E
  misc_feature (11)..(11): Xaa = L, I, or V
  misc_feature (12)..(12): Xaa = V, L, M, or K
  misc_feature (13)..(13): Xaa = K, A, Q, or R
  misc_feature (15)..(15): Xaa = S or G
  misc_feature (16)..(16): Xaa = Q, G, A, T, V, or E
  misc_feature (17)..(17): Xaa = S, T, L, or P
  misc_feature (18)..(18): Xaa = L, V, or E
  misc_feature (19)..(19): Xaa = S or K
  misc_feature (20)..(20): Xaa = L, I, or M
  misc_feature (21)..(21): Xaa = T or S
  misc_feature (23)..(23): Xaa = T, S, A, or K
  misc_feature (24)..(24): Xaa = V, F, A, G, or T
  misc_feature (25)..(25): Xaa = T, S, or F
  misc_feature (27)..(27): Xaa = Y, F, or I
  misc_feature (28)..(28): Xaa = S, I, T, N, or D
  misc_feature (29)..(29): Xaa = I, S, L, or F
  misc_feature (30)..(30): Xaa = S, or is absent
  misc_feature (31)..(31): Xaa = T, or is absent
  misc_feature (32)..(32): Xaa = S, K, or T
  misc_feature (33)..(33): Xaa = D, G, S, A, or Y, or is absent
  misc_feature (34)..(34): Xaa = Y, S, T, I, F, D, or H
  misc_feature (35)..(35): Xaa = A, G, Y, T, W, V, D, F, H, or S
  misc_feature (36)..(36): Xaa = W, V, M, I, or L
  misc_feature (37)..(37): Xaa = N, H, S, F, Y, V, or E
  misc_feature (39)..(39): Xaa = I or V
  misc_feature (40)..(40): Xaa = R or K
  misc_feature (42)..(42): Xaa = F, P, T, R, S, or A
  misc_feature (43)..(43): Xaa = P, S, T, R, or H
  misc_feature (44)..(44): Xaa = G, R, E, or A
  misc_feature (45)..(45): Xaa = N, K, Q, H, or R
  misc_feature (46)..(46): Xaa = K, G, R, S, or D
  misc_feature (48)..(48): Xaa = E, D, or K
  misc_feature (50)..(50): Xaa = M, L, V, or I
  misc_feature (51)..(51): Xaa = G, V, or A
  misc_feature (52)..(52): Xaa = Y, V, H, T, F, R, W, or E
  misc_feature (53)..(53): Xaa = I or V
  misc_feature (54)..(54): Xaa = S, W, Y, D, L, or N
  misc_feature (55)..(55): Xaa = P, T, or S, or is absent
  misc_feature (56)..(56): Xaa = Y, S, W, D, A, E, or G
  misc_feature (57)..(57): Xaa = S, D, G, N, Y, or T
  misc_feature (58)..(58): Xaa = G, D, or S
  misc_feature (59)..(59): Xaa = S, A, D, R, T, N, E, or Y
  misc_feature (60)..(60): Xaa = T, K, Y, N, I, S, or P
  misc_feature (61)..(61): Xaa = S, T, R, V, K, N, D, F, H, or G
  misc_feature (63)..(63): Xaa = N, Y, D,  or A
  misc_feature (64)..(64): Xaa = P, S, E, Q, H, or D
  misc_feature (65)..(65): Xaa = D, or is absent
  misc_feature (66)..(66): Xaa = S, A, T, K, N, or D
  misc_feature (67)..(67): Xaa = L, V, or F
  misc_feature (68)..(68): Xaa = K, Q, T, or R
  misc_feature (69)..(69): Xaa = S, G, or D
  misc_feature (70)..(70): Xaa = R or K
  misc_feature (71)..(71): Xaa = I, N, F, L, or A
  misc_feature (72)..(72): Xaa = S, T, N, or A
  misc_feature (73)..(73): Xaa = I, L, M, or F
  misc_feature (74)..(74): Xaa = T, S, or A
  misc_feature (75)..(75): Xaa = R, K, A, V, or L
  misc_feature (76)..(76): Xaa = D, H, G, or E
  misc_feature (77)..(77): Xaa = T, N, V, A, or K
  misc_feature (78)..(78): Xaa = S or A
  misc_feature (79)..(79): Xaa = K, T, S, or A
  misc_feature (80)..(80): Xaa = N, S, R, or T
  misc_feature (81)..(81): Xaa = Q, N, A, I, or T
  misc_feature (82)..(82): Xaa = F, V, L, or A
  misc_feature (83)..(83): Xaa = F, Y, or H
  misc_feature (84)..(84): Xaa = L, M, or I
  misc_feature (85)..(85): Xaa = Q, K, E, or D
  misc_feature (86)..(86): Xaa = L, M, or I
  misc_feature (87)..(87): Xaa = N, T, S, G, A, or L
  misc_feature (88)..(88): Xaa = S, R, or N
  misc_feature (89)..(89): Xaa = V or L
  misc_feature (90)..(90): Xaa = T, Q, D, K, or S
  misc_feature (91)..(91): Xaa = T, S, or N
  misc_feature (92)..(92): Xaa = E, D, A, or G
  misc_feature (94)..(94): Xaa = T or S
  misc_feature (96)..(96): Xaa = T, M, I, L, or V
  misc_feature (98)..(98): Xaa = Y or F
  misc_feature (100)..(100): Xaa = T, or A, or is absent
  misc_feature (101)..(101): Xaa = A, T, G, S, or R
  misc_feature (102)..(102): Xaa = R, S, L, or E, or is absent
  misc_feature (103)..(103): Xaa = H, E, R, G, or T, or is absent
  misc_feature (104)..(104): Xaa = G, N, S, D, W, L, K, I, T, or V, or is absent
  misc_feature (105)..(105): Xaa = R, Y, N, L, F, T, V, A, or S, or is absent
  misc_feature (106)..(106): Xaa = D, E, Y, F, Q, S, L, V, or T, or is absent
  misc_feature (107)..(107): Xaa = G, D, S, T, R, or A
  misc_feature (108)..(108): Xaa = Y, I, S, G, T, D, F, M, P, or N, or is absent
  misc_feature (109)..(109): Xaa = L, H, S, V, I, N, or K, or is absent
  misc_feature (110)..(110): Xaa = Y, W, V, T, or G, or is absent
  misc_feature (111)..(111): Xaa = G, E, T, or V, or is absent
  misc_feature (112)..(112): Xaa = Y, A, S, N, G, V, or F, or is absent
  misc_feature (113)..(113): Xaa = A, D, P, R, Y, or S, or is absent
  misc_feature (114)..(114): Xaa = Y, A, or W, or is absent
  misc_feature (115)..(115): Xaa = Y, or is absent
  misc_feature (116)..(116): Xaa = A, or is absent
  misc_feature (117)..(117): Xaa = F, M, or L
  misc_feature (118)..(118): Xaa = D or A
  misc_feature (119)..(119): Xaa = Y or V
  misc_feature (122)..(122): Xaa = Q, P, or A
  misc_feature (125)..(125): Xaa = T, S, L, or P
  misc_feature (126)..(126): Xaa = L, or V, or is absent
  misc_feature (127)..(127): Xaa = T, or is absent
  misc_feature (128)..(128): Xaa = V, or is absent
  misc_feature (129)..(129): Xaa = S, or is absent
XXXLXZXGXXXXXPXXXXXXXCXXXGXXXXXXXXXXXWXXQXXXXXLXWXXXXXXXXXXXXYXXXXXXXXXXXXXXXXXXXXXXXXXXXXXXBXAXYXCXXXXXXXXXXXXXXXXXXXXWGXGTXXXXX

SEQ ID NO: 150 - protein, 115 aa, Artificial Sequence
  Consensus Sequence of the Light Chain Sequences of SEQ ID NOs 25-48
  misc_feature (1)..(1): Xaa = D or E
  misc_feature (2)..(2): Xaa = I, N, or V
  misc_feature (3)..(3): Xaa = Q, K, or V
  misc_feature (4)..(4): Xaa = M or L
  misc_feature (7)..(7): Xaa = T, S, or A
  misc_feature (8)..(8): Xaa = T, P, A, Q, or H
  misc_feature (9)..(9): Xaa = S, A, P, L, or K
  misc_feature (10)..(10): Xaa = S, I, L, T, or F
  misc_feature (11)..(11): Xaa = L, Q, M, or I
  misc_feature (12)..(12): Xaa = S, Y, P, A, or T
  misc_feature (13)..(13): Xaa = A, V, M, or T
  misc_feature (14)..(14): Xaa = S or T
  misc_feature (15)..(15): Xaa = L, R, P, I, V, or A
  misc_feature (16)..(16): Xaa = E or G
  misc_feature (17)..(17): Xaa = D, E, G, or Q
  misc_feature (18)..(18): Xaa = R, K, S, P, or Q
  misc_feature (19)..(19): Xaa = V or A
  misc_feature (20)..(20): Xaa = T or S
  misc_feature (21)..(21): Xaa = I, M, V, or L
  misc_feature (22)..(22): Xaa = S, N, or T
  misc_feature (24)..(24): Xaa = S, K, T, or R
  misc_feature (25)..(25): Xaa = A or S
  misc_feature (26)..(26): Xaa = S, N, or G
  misc_feature (27)..(27): Xaa = Q or S
  misc_feature (28)..(28): Xaa = G, D, S, or N
  misc_feature (29)..(29): Xaa = I, V, or L
  misc_feature (30)..(30): Xaa = R, S, N, K, L, V, G, or D, or is absent
  misc_feature (31)..(31): Xaa = S, H, D, Y, N, or T, or is absent
  misc_feature (32)..(32): Xaa = N, D, S, or A, or is absent
  misc_feature (33)..(33): Xaa = S, D, N, or G, or is absent
  misc_feature (34)..(34): Xaa = G, N, or D, or is absent
  misc_feature (35)..(35): Xaa = Q, or is absent
  misc_feature (36)..(36): Xaa = N, I, or K, or is absent
  misc_feature (37)..(37): Xaa = T, N, or S, or is absent
  misc_feature (38)..(38): Xaa = Y, F, or N, or is absent
  misc_feature (39)..(39): Xaa = L, M, or V
  misc_feature (40)..(40): Xaa = N, S, H, Y, or A
  misc_feature (42)..(42): Xaa = Y, F, or L
  misc_feature (43)..(43): Xaa = Q or L
  misc_feature (45)..(45): Xaa = K or R
  misc_feature (46)..(46): Xaa = P or S
  misc_feature (47)..(47): Xaa = D, W, G, R, or H
  misc_feature (48)..(48): Xaa = G, V, K, S, Q, or E
  misc_feature (49)..(49): Xaa = T, S, or P
  misc_feature (50)..(50): Xaa = V or P
  misc_feature (51)..(51): Xaa = K, Q, H, or R
  misc_feature (52)..(52): Xaa = L, T, P, R, or A
  misc_feature (53)..(53): Xaa = L or W
  misc_feature (55)..(55): Xaa = F, Y, or K
  misc_feature (56)..(56): Xaa = F, Y, S, L, R, K, W, G, or A
  misc_feature (57)..(57): Xaa = T, A, M, V, or E
  misc_feature (58)..(58): Xaa = S or T
  misc_feature (59)..(59): Xaa = T, S, I, N, K, Y, or Q
  misc_feature (60)..(60): Xaa = L, R, or S
  misc_feature (61)..(61): Xaa = H, A, D, F, E, Y, or I
  misc_feature (62)..(62): Xaa = S, D, or T
  misc_feature (64)..(64): Xaa = V or I
  misc_feature (66)..(66): Xaa = S, T, A, D, or N
  misc_feature (68)..(68): Xaa = F or L
  misc_feature (69)..(69): Xaa = S or T
  misc_feature (75)..(75): Xaa = T, Q, or S
  misc_feature (76)..(76): Xaa = D, E, S, or A
  misc_feature (77)..(77): Xaa = Y, F, or L
  misc_feature (78)..(78): Xaa = S or T
  misc_feature (80)..(80): Xaa = T, R, K, S, or N
  misc_feature (82)..(82): Xaa = S, N, or H
  misc_feature (83)..(83): Xaa = N, S, R, K, or P
  misc_feature (84)..(84): Xaa = L, M, or V
  misc_feature (85)..(85): Xaa = E, A, K, or Q
  misc_feature (86)..(86): Xaa = P, S, A, or E
  misc_feature (87)..(87): Xaa = E or D
  misc_feature (89)..(89): Xaa = I, T, A, V, or L
  misc_feature (90)..(90): Xaa = A or G
  misc_feature (91)..(91): Xaa = T, V, E, D, or I
  misc_feature (93)..(93): Xaa = Y or F
  misc_feature (95)..(95): Xaa = H, Q, L, M, W, or S
  misc_feature (96)..(96): Xaa = Q or N
  misc_feature (97)..(97): Xaa = Y, H, W, G, S, or D
  misc_feature (98)..(98): Xaa = S, G, H, L, T, Y, F, or N
  misc_feature (99)..(99): Xaa = K, E, R, I, H, or S
  misc_feature (100)..(100): Xaa = L, F, S, N, Y, V, or D
  misc_feature (101)..(101): Xaa = P, or is absent
  misc_feature (102)..(102): Xaa = Y, F, W, L, or R, or is absent
  misc_feature (106)..(106): Xaa = G, S, A, W, or D
  misc_feature (111)..(111): Xaa = E, or is absent
  misc_feature (112)..(112): Xaa = L or I, or is absent
  misc_feature (113)..(113): Xaa = K, or is absent
  misc_feature (114)..(114): Xaa = R, or is absent
  misc_feature (115)..(115): Xaa = T, or is absent
XXXXTQXXXXXXXXXXXXXXXXCXXXXXXXXXXXXXXXXXWXXQXXXXXXXXXIXXXXXXXXGXPXRXXGSGSGXXXXLXIXXXXXXDXXXYXCXXXXXXXXTFGXGTKLXXXXX

SEQ ID NO: 151 - protein, 121 aa, Artificial Sequence
  Consensus Sequence of the Heavy Chain Sequences of SEQ ID NOs 49-61
  misc_feature (9)..(9): Xaa = A, P, T, or G
  misc_feature (10)..(10): Xaa = E, G, or D
  misc_feature (11)..(11): Xaa = L, R, or S
  misc_feature (12)..(12): Xaa = V, M, or K
  misc_feature (13)..(13): Xaa = R, K, or Q
  misc_feature (16)..(16): Xaa = S, A, E, G, or R
  misc_feature (17)..(17): Xaa = S or T
  misc_feature (18)..(18): Xaa = V or L
  misc_feature (20)..(20): Xaa = I, M, or L
  misc_feature (23)..(23): Xaa = K or A
  misc_feature (24)..(24): Xaa = V or A
  misc_feature (25)..(25): Xaa = S or T
  misc_feature (27)..(27): Xaa = Y, S, or F
  misc_feature (28)..(28): Xaa = T, S, or I
  misc_feature (29)..(29): Xaa = F or L
  misc_feature (30)..(30): Xaa = S or T
  misc_feature (31)..(31): Xaa = N, R, S, Y, D, or T
  misc_feature (32)..(32): Xaa = Y or H
  misc_feature (33)..(33): Xaa = W, V, G, S, Y, or T
  misc_feature (34)..(34): Xaa = M, I, or L
  misc_feature (35)..(35): Xaa = N, H, E, Y, F, or S
  misc_feature (37)..(37): Xaa = V or F
  misc_feature (38)..(38): Xaa = K or R
  misc_feature (40)..(40): Xaa = R, K, A, or T
  misc_feature (42)..(42): Xaa = G or E
  misc_feature (43)..(43): Xaa = Q, H, or K
  misc_feature (44)..(44): Xaa = G or R
  misc_feature (46)..(46): Xaa = E or K
  misc_feature (48)..(48): Xaa = I, M, or V
  misc_feature (49)..(49): Xaa = G or A
  misc_feature (50)..(50): Xaa = Q, Y, E, W, or T
  misc_feature (52)..(52): Xaa = Y, N, L, or S
  misc_feature (53)..(53): Xaa = P, T, D, or S
  misc_feature (54)..(54): Xaa = G, Y, or E
  misc_feature (55)..(55): Xaa = D, N, S, T, or G
  misc_feature (56)..(56): Xaa = D, G, R, or T
  misc_feature (57)..(57): Xaa = S, G, N, E, or Y
  misc_feature (58)..(58): Xaa = T, P, or S
  misc_feature (59)..(59): Xaa = Y, K, N, or T
  misc_feature (61)..(61): Xaa = N, A, P, or L
  misc_feature (62)..(62): Xaa = G, E, or D
  misc_feature (63)..(63): Xaa = K, D, or S
  misc_feature (64)..(64): Xaa = F or V
  misc_feature (65)..(65): Xaa = K, R, or Q
  misc_feature (66)..(66): Xaa = D, G, or A
  misc_feature (67)..(67): Xaa = K or R
  misc_feature (68)..(68): Xaa = A or F
  misc_feature (69)..(69): Xaa = K, T, or A
  misc_feature (70)..(70): Xaa = M, L, F, V, or I
  misc_feature (71)..(71): Xaa = T or S
  misc_feature (72)..(72): Xaa = A, S, L, or R
  misc_feature (73)..(73): Xaa = D or E
  misc_feature (74)..(74): Xaa = K, T, or N
  misc_feature (75)..(75): Xaa = S or A
  misc_feature (76)..(76): Xaa = S, T, A, or K
  misc_feature (77)..(77): Xaa = S, N, or T
  misc_feature (78)..(78): Xaa = T, N, or K
  misc_feature (79)..(79): Xaa = A, L, or V
  misc_feature (80)..(80): Xaa = Y or N
  misc_feature (81)..(81): Xaa = I, M, or L
  misc_feature (83)..(83): Xaa = L, I, M, or T
  misc_feature (84)..(84): Xaa = S, R, T, or N
  misc_feature (85)..(85): Xaa = S or N
  misc_feature (87)..(87): Xaa = T or K
  misc_feature (88)..(88): Xaa = S or N
  misc_feature (89)..(89): Xaa = E or D
  misc_feature (91)..(91): Xaa = S, M, or T
  misc_feature (93)..(93): Xaa = V, T, M, I, or L
  misc_feature (94)..(94): Xaa = Y or H
  misc_feature (95)..(95): Xaa = F, Y, or H
  misc_feature (97)..(97): Xaa = A, T, or V
  misc_feature (99)..(99): Xaa = L, R, T, K, or D, or is absent
  misc_feature (100)..(100): Xaa = R, G, W, H, or N
  misc_feature (101)..(101): Xaa = D, or is absent
  misc_feature (102)..(102): Xaa = P, F, E, G, Y, or A
  misc_feature (103)..(103): Xaa = L, R, T, N, D, or Y
  misc_feature (104)..(104): Xaa = F, Y, R, W, S, G, or V, or is absent
  misc_feature (105)..(105): Xaa = Y, R, G, W, L, or S
  misc_feature (106)..(106): Xaa = A, Y, N, G, or S, or is absent
  misc_feature (107)..(107): Xaa = M, F, V, Y, S, G, or R
  misc_feature (108)..(108): Xaa = D, A, Y, or G
  misc_feature (109)..(109): Xaa = S, M, or F, or is absent
  misc_feature (110)..(110): Xaa = A, or D, or is absent
  misc_feature (111)..(111): Xaa = Y or V
  misc_feature (114)..(114): Xaa = Q or A
  misc_feature (117)..(117): Xaa = S, T, or L
  misc_feature (118)..(118): Xaa = V or L
  misc_feature (121)..(121): Xaa = S, or is absent
EVQLQESGXXXXXPGXXXKXSCXXXGXXXXXXXXXWXXQXPXXXLXWXXXIXXXXXXXXYXXXXXXXXXXXXXXXXXXXXXZXXXLXXXDXAXXXCXRXXXXXXXXXXXXXWGXGTXXTVX

SEQ ID NO: 152 - protein, 114 aa, Artificial Sequence
  Consensus Sequence of the Light Chain Sequences of SEQ ID NOs 62-74
  misc_feature (1)..(1): Xaa = D or E
  misc_feature (2)..(2): Xaa = I, V, or N
  misc_feature (3)..(3): Xaa = Q or V
  misc_feature (4)..(4): Xaa = M, L, or I
  misc_feature (7)..(7): Xaa = S or T
  misc_feature (9)..(9): Xaa = S, L, or A
  misc_feature (10)..(10): Xaa = S, T, or I
  misc_feature (11)..(11): Xaa = L or M
  misc_feature (12)..(12): Xaa = A, S, or Y
  misc_feature (13)..(13): Xaa = M, V, or A
  misc_feature (14)..(14): Xaa = S or T
  misc_feature (15)..(15): Xaa = V, I, L, or P
  misc_feature (17)..(17): Xaa = Q, E, or D
  misc_feature (18)..(18): Xaa = K, P, R, or T
  misc_feature (19)..(19): Xaa = V or A
  misc_feature (20)..(20): Xaa = T or S
  misc_feature (21)..(21): Xaa = M or I
  misc_feature (22)..(22): Xaa = S or T
  misc_feature (24)..(24): Xaa = K, R, or S
  misc_feature (25)..(25): Xaa = S, A, or V
  misc_feature (27)..(27): Xaa = Q, E, or S
  misc_feature (28)..(28): Xaa = S, T, N, or D
  misc_feature (29)..(29): Xaa = L, V, or I, or is absent
  misc_feature (30)..(30): Xaa = L, or V, or is absent
  misc_feature (31)..(31): Xaa = N, D, or G, or is absent
  misc_feature (32)..(32): Xaa = S, or Y, or is absent
  misc_feature (33)..(33): Xaa = S, D, or Y, or is absent
  misc_feature (34)..(34): Xaa = N, or G, or is absent
  misc_feature (35)..(35): Xaa = Q, or is absent
  misc_feature (36)..(36): Xaa = K, E, D, N, Y, or S, or is absent
  misc_feature (37)..(37): Xaa = N, T, S, or D
  misc_feature (38)..(38): Xaa = Y, H, F, or N
  misc_feature (39)..(39): Xaa = L or M
  misc_feature (40)..(40): Xaa = A, N, H, or S
  misc_feature (42)..(42): Xaa = Y, L, or F
  misc_feature (43)..(43): Xaa = Q or L
  misc_feature (45)..(45): Xaa = K or R
  misc_feature (46)..(46): Xaa = P or Q
  misc_feature (47)..(47): Xaa = G, D, or W
  misc_feature (48)..(48): Xaa = Q, K, G, or T
  misc_feature (49)..(49): Xaa = S, P, or T
  misc_feature (50)..(50): Xaa = P or V
  misc_feature (51)..(51): Xaa = K or Q
  misc_feature (52)..(52): Xaa = L, R, or T
  misc_feature (53)..(53): Xaa = L, R, or W
  misc_feature (54)..(54): Xaa = V or I
  misc_feature (56)..(56): Xaa = F, L, A, R, Y, or S
  misc_feature (57)..(57): Xaa = A, V, or T
  misc_feature (58)..(58): Xaa = S, P, or T
  misc_feature (59)..(59): Xaa = T, E, K, N, D, or S
  misc_feature (60)..(60): Xaa = R or L
  misc_feature (61)..(61): Xaa = E, D, A, or H
  misc_feature (62)..(62): Xaa = S or D
  misc_feature (64)..(64): Xaa = V or I
  misc_feature (66)..(66): Xaa = D, A, or S
  misc_feature (68)..(68): Xaa = F or L
  misc_feature (69)..(69): Xaa = I, T, or S
  misc_feature (71)..(71): Xaa = S or R
  misc_feature (74)..(74): Xaa = G or R
  misc_feature (75)..(75): Xaa = T or Q
  misc_feature (76)..(76): Xaa = D, Q, H, or S
  misc_feature (77)..(77): Xaa = F or Y
  misc_feature (78)..(78): Xaa = T or S
  misc_feature (80)..(80): Xaa = T, K, or N
  misc_feature (82)..(82): Xaa = S, H, or N
  misc_feature (83)..(83): Xaa = S, R, P, or N
  misc_feature (84)..(84): Xaa = V, A, L, or M
  misc_feature (86)..(86): Xaa = A, E, S, or P
  misc_feature (87)..(87): Xaa = E or D
  misc_feature (89)..(89): Xaa = L, V, A, F, or T
  misc_feature (90)..(90): Xaa = A or G
  misc_feature (91)..(91): Xaa = D, I, V, T, or S
  misc_feature (93)..(93): Xaa = F or Y
  misc_feature (95)..(95): Xaa = Q, W, or L
  misc_feature (96)..(96): Xaa = Q or H
  misc_feature (97)..(97): Xaa = H, G, E, S, V, F, Y, or R
  misc_feature (98)..(98): Xaa = Y, T, N, W, S, or G
  misc_feature (99)..(99): Xaa = S, H, G, E, or K
  misc_feature (100)..(100): Xaa = A, F, D, T, L, S, or Y
  misc_feature (102)..(102): Xaa = P, R, W, F, Y, or L
  misc_feature (106)..(106): Xaa = A, G, or S
  misc_feature (112)..(112): Xaa = I or L
  misc_feature (113)..(113): Xaa = K, or is absent
  misc_feature (114)..(114): Xaa = R, or is absent
XXXXTQXPXXXXXXXGXXXXXXCXXSXXXXXXXXXXXXXXWXXQXXXXXXXXXXYXXXXXXXGXPXRXXGXGSXXXXXLXIXXXZXXDXXXYXCXXXXXXPXTFGXGTKLEXXX

SEQ ID NO: 153 - protein, 22 aa, Artificial Sequence
  Consensus sequence of the heavy chain CDRs of SEQ ID NOs 75-98
  misc_feature (2)..(2): Xaa = A, S, or T, or is absent
  misc_feature (3)..(3): Xaa = R, A, or S, or is absent
  misc_feature (4)..(4): Xaa = G, S, D, E, R, H, or W, or is absent
  misc_feature (5)..(5): Xaa = R, H, N, V, G, L, T, I, S, or D, or is absent
  misc_feature (6)..(6): Xaa = D, Y, A, G, F, R, L, Q, V, T, or N, or is absent
  misc_feature (7)..(7): Xaa = T, L, S, R, Y, or F, or is absent
  misc_feature (8)..(8): Xaa = A, or S, or is absent
  misc_feature (9)..(9): Xaa = S, or is absent
  misc_feature (10)..(10): Xaa = M, or is absent
  misc_feature (11)..(11): Xaa = T, I, R, or D, or is absent
  misc_feature (12)..(12): Xaa = D, K, T, L, G, or V, or is absent
  misc_feature (13)..(13): Xaa = G, N, T, L, F, or V, or is absent
  misc_feature (14)..(14): Xaa = Y, W, V, L, or A, or is absent
  misc_feature (15)..(15): Xaa = L, D, G, F, Y, R, V, E, or T, or is absent
  misc_feature (16)..(16): Xaa = Y, N, S, H, R, G, K, F, or A, or is absent
  misc_feature (17)..(17): Xaa = Y, H, D, W, A, P, N, or G, or is absent
  misc_feature (18)..(18): Xaa = D, A, P, or R, or is absent
  misc_feature (19)..(19): Xaa = F, M, or L
  misc_feature (20)..(20): Xaa = D or A
  misc_feature (21)..(21): Xaa = Y or V
CXXXXXXXXXXXXXXXXXXXXW

SEQ ID NO: 154 - protein, 11 aa, Artificial Sequence
  Consensus sequence of the light chain CDRs of SEQ ID NOs 99-122
  misc_feature (2)..(2): Xaa = H, W, M, L, W, or S
  misc_feature (3)..(3): Xaa = Q or N
  misc_feature (4)..(4): Xaa = Y, W, D, G, H, or S
  misc_feature (5)..(5): Xaa = S, Y, F, N, H, L, G, or T
  misc_feature (6)..(6): Xaa = K, S, I, R, E, or H
  misc_feature (7)..(7): Xaa = L, F, Y, N, S, D, or V
  misc_feature (8)..(8): Xaa = P, or is absent
  misc_feature (9)..(9): Xaa = Y, W, F, L, or R, or is absent
CXXXXXXXXTF

SEQ ID NO: 155 - protein, 18 aa, Artificial Sequence
  Consensus sequence of the heavy chain CDRs of SEQ ID NOs 123-135
  misc_feature (2)..(2): Xaa = A, T, or V
  misc_feature (3)..(3): Xaa = R, or is absent
  misc_feature (4)..(4): Xaa = K, T, or D, or is absent
  misc_feature (5)..(5): Xaa = R, H, L, or N, or is absent
  misc_feature (6)..(6): Xaa = R, G, or Y, or is absent
  misc_feature (7)..(7): Xaa = P, N, E, D, or A, or is absent
  misc_feature (8)..(8): Xaa = L, S, R, T, G, N, or Y
  misc_feature (9)..(9): Xaa = F, Y, W, or L
  misc_feature (10)..(10): Xaa = Y, V, or F, or is absent
  misc_feature (11)..(11): Xaa = Y, F, G, S, or L, or is absent
  misc_feature (12)..(12): Xaa = A, N, S, G, R, or V, or is absent
  misc_feature (13)..(13): Xaa = M, V, R, or L, or is absent
  misc_feature (14)..(14): Xaa = D, Y, G, or T
  misc_feature (15)..(15): Xaa = Y, S, or F
  misc_feature (16)..(16): Xaa = A, D, or M, or is absent
  misc_feature (17)..(17): Xaa = Y, V, or S, or is absent
  misc_feature (18)..(18): Xaa = W or G
CXXXXXXXXXXXXXXXXX

SEQ ID NO: 156 - protein, 14 aa, Artificial Sequence
  Consensus sequence of the light chain CDRs of SEQ ID NOs 136-148
  misc_feature (2)..(2): Xaa = Q, W, or L, or is absent
  misc_feature (4)..(4): Xaa = G, E, Y, H, S, or R, or is absent
  misc_feature (5)..(5): Xaa = T, S, G, or N, or is absent
  misc_feature (6)..(6): Xaa = H, or is absent
  misc_feature (7)..(7): Xaa = V or F, or is absent
  misc_feature (8)..(8): Xaa = Y or W, or is absent
  misc_feature (9)..(9): Xaa = S, G, K, or E, or is absent
  misc_feature (10)..(10): Xaa = A, T, L, S, D, or Y, or is absent
  misc_feature (12)..(12): Xaa = P, W, R, F, L, or Y
CXQXXXXXXXPXTF